SEQUENCE LISTING (9 sequences)

SEQ ID NO: 1 - DNA, 24 bp, Artificial Sequence
  Forward primer for human RBP1
tttattgggtattggaagatgttg

SEQ ID NO: 2 - DNA, 25 bp, Artificial Sequence
  Reverse primer for human RBP1
tccaatctacaacctaaaaactacc

SEQ ID NO: 3 - DNA, 22 bp, Artificial Sequence
  Forward primer for human RBP1
ggtattggaagatgttggttaa

SEQ ID NO: 4 - DNA, 20 bp, Artificial Sequence
  Forward primer for human IDH1
gcgtcaaatgtgccactatc

SEQ ID NO: 5 - DNA, 22 bp, Artificial Sequence
  Reverse primer for human IDH1
gcaaaatcacattattgccaac

SEQ ID NO: 6 - DNA, 21 bp, Artificial Sequence
  Forward primer for human IDH2
ctcacagagttcaagctgaag

SEQ ID NO: 7 - DNA, 21 bp, Artificial Sequence
  Reverse primer for human IDH2
ctgtggccttgtactgcagag

SEQ ID NO: 8 - DNA, 20 bp, Artificial Sequence
  Forward primer for human RBP1
caactggctccagtcactcc

SEQ ID NO: 9 - DNA, 20 bp, Artificial Sequence
  Reverse primer for human RBP1
tgcacgatctctttgtctgg

(19) United States
(12) Patent Application Publication
(10) Pub. No.: US 2014/0343145 A1
(43) Pub. Date: Nov. 20, 2014

(54) RBP1 as a Molecular Biomarker for Predicting Survival and Response to Treatment in Glioma

(71) Applicant: The Regents of the University of California, Oakland, CA (US)

(72) Inventors: Arthur P. Chou, Playa Del Rey, CA (US); Albert Lai, Glendale, CA (US); Reshmi Chowdhury, Los Angeles, CA (US); Weidong Chen, Los Angeles, CA (US)

(73) Assignee: THE REGENTS OF THE UNIVERSITY OF CALIFORNIA, Oakland, CA (US)

(21) Appl. No.: 14/360,734

(22) PCT Filed: Nov. 27, 2012
(86) PCT No.: PCT/US12/66673

§ 371 Date: May 27, 2014

Related U.S. Application Data
(60) Provisional application No. 61/564,497, filed on Nov. 29, 2011.

(51) Int. Cl. C12Q 1/68

(52) U.S. Cl. CPC C12Q 1/6886; C12Q 2600/118; C12Q 2600/154
USPC 514/559; 435/6.11

(57) ABSTRACT

Disclosed herein are methods for detecting the presence of an isocitrate dehydrogenase mutation in a sample from a subject. Also disclosed are methods diagnosing and treating subjects having a cancer including determining whether the cancer may likely be treated with one or more retinoids.

### CROSS-REFERENCE TO RELATED APPLICATIONS

This application claims the benefit of U.S. Patent Application Ser. No. 61/564,497, filed 29 Nov. 2011, which is herein incorporated by reference in its entirety.

### ACKNOWLEDGEMENT OF GOVERNMENT SUPPORT

This invention was made with Government support of Grant No. K08 CA124479, awarded by the National Institutes of Health. The Government has certain rights in this invention.

### REFERENCE TO A SEQUENCE LISTING SUBMITTED VIA EFS-WEB

The content of the ASCII text file of the sequence listing named “20121119_{—}034044_{—}096WO1_seq_ST25” which is 2.07 kb in size was created on 19 Nov. 2012 and electronically submitted via EFS-Web herewith the application is incorporated herein by reference in its entirety.

### BACKGROUND OF THE INVENTION

1. Field of the Invention

The present invention generally relates to hypermethylation in the retinol-binding protein 1 (RBP1) promoter and methods of diagnosing and/or treating subjects having hypermethylated RBP1 promoters.

2. Description of the Related Art

Diffuse gliomas represent the most common type of adult primary brain cancer, affecting as many as 20,000 patients per year in the United States. A mutation in the enzyme isocitrate dehydrogenase 1 (IDH1) was found in secondary glioblastomas in the year 2008 and has since also been reported in acute myelogenous leukemia (Parsons D W, et al. Science 2008; 321(5897):1807-12; Gross S, et al. J Exp Med 2010; 207(2):339-44; Ward P S, et al. Cancer Cell 2010; 17(3):225-34). IDH1 mutations are uniformly heterozygous at residue R132, and accumulating evidence indicates that it is likely an early event in the development of glioma (Lai A, et al. J Clin Oncol 2011; 29(34):4482-90; Watanabe T, et al. Am J Pathol 2009; 174(4):1149-53). With lesser frequency, mutations in isocitrate dehydrogenase 2 (IDH2) have also been found in gliomas (Yan H, et al. N Engl J Med 2009; 360(8):765-73), but the role of IDH1 and IDH2 mutations in glioma formation has not been elucidated. Importantly, it was reported that the IDH1 mutant protein preferentially catalyzes the formation of 2-hydroxyglutarate (2-HG) (Dang L, et al. Nature 2009; 462(7274):739-44), a metabolite potentially contributing to gliomagenesis.

Also, a number of studies have confirmed that IDH1 mutant gliomas harbor hypermethylation of hundreds of various genes (Noushmehr H, et al. Cancer Cell 2010; 17(5):510-22; Christensen B C, et al. J Natl Cancer Inst 2011; 103(2):143-53; Turcan S, et al. Nature 2012; 483(7390):479-83; Laffaire J, et al. Neuro Oncol 2011; 13(1):84-98). Such hypermethylation is present in low grade gliomas and high grade glioblastoma (GBM). Thus, hypermethylation of one or more genes has been hypothesized to be associated with the early stages of gliomagenesis. However, it is unknown whether there is one particular hypermethylation profile of a specific gene that can be used reliably to identify a glioma as having an IDH1 mutation. Further, it is unknown whether such a particular hypermethylation profile would merely be the result of gliomagenesis or whether it contribute to the formation of gliomas via silencing one or more tumor suppressor genes.

### SUMMARY OF THE INVENTION

In some embodiments, the present invention is directed to assays for detecting the presence of an isocitrate dehydrogenase mutation in a sample obtained from a subject which comprise determining the methylation status of all or part of the retinol-binding protein 1 (RBP1) promoter in the sample, wherein the presence of the isocitrate dehydrogenase mutation is indicated where the methylation status is hypermethylated. In some embodiments, the assays further comprise characterizing the methylation status as hypermethylated where all or part of the RBP1 promoter is significantly more methylated than that of the corresponding part of a standard which is a methylation profile of a wild type RBP1 promoter or a consensus methylation profile of the RBP1 promoters obtained from a plurality of normal subjects. In some embodiments, the methylation status is characterized as being hypermethylated where the overall methylation of the methylation profile is methylated by 50% or more than that of the standard. In some embodiments, the RBP1 promoter is located on chromosome 3 at 140740839-140741418. In some embodiments, the part of the RBP1 promoter comprises, consists essentially of, or consists of CpG sites 5-25 of the CpG island located on chromosome 3 at 140740839-140741418. In some embodiments, the part of the RBP1 promoter comprises, consists essentially of, or consists of one or more of the CpG sites of the RBP1 promoter. In some embodiments, the part of the RBP1 promoter comprises, consists essentially of, or consists of at least 3, preferably at least 5, more preferably at least 10 CpG sites selected from the group consisting of CpG sites 14-24, 26-31, 39-45 and 59 of the CpG island located on chromosome 3 at 140740839-140741418. In some embodiments, the part of the RBP1 promoter comprises, consists essentially of, or consists of at least 6, preferably at least 10, more preferably at least 15 CpG sites selected from the group consisting of CpG sites 1-5, 14-24, 26-31, 39-45 and 59 of the CpG island located on chromosome 3 at 140740839-140741418. In some embodiments, the part of the RBP1 promoter comprises, consists essentially of, or consists of at least 10, preferably at least 15, more preferably at least 20 CpG sites selected from the group consisting of CpG sites 1-5, 14-24, 26-31, 39-45 and 59-62 of the CpG island located on chromosome 3 at 140740839-140741418. In some embodiments, the methylation status is characterized as being hypermethylated where the overall methylation of the all or part of the RBP1 promoter is methylated by 50% or more than that of the corresponding part of a standard which is a methylation profile of a wild type RBP1 promoter or a consensus methylation profile of the RBP1 promoters obtained from a plurality of normal subjects. In some embodiments, when one or more of the CpG sites 15, 21, 24, 29, 30, 40, 44 or 45 of the CpG island located on chromosome 3 at 140740839-140741418 are methylated, the methylation status is designated as hypermethylated. In some embodiments, the assay comprises modifying all or part of the retinol-binding protein 1 (RBP1) promoter in the sample to make the methylated cytosines of CpG dinucleotides distinguishable from the unmethylated cytosines of CpG dinucleotides. In some embodiments, the assay comprises subjecting all or part of the retinol-binding protein 1 (RBP1) promoter in the sample to (a) a bisulfite that converts the unmethylated cytosines to uracils, (b) a restriction enzyme that selectively cleaves the unmethylated cytosines, (c) a label specific for either the unmethylated cytosines or the methylated cytosines, or (d) a combination thereof. In some embodiments, the methylation status is determined using reduced representation bisulfite sequencing (RRBS). In some embodiments, the assays further comprise designating the presence of the isocitrate dehydrogenase mutation where the methylation status is hypermethylated. In some embodiments, the isocitrate dehydrogenase mutation is an isocitrate dehydrogenase 1 (IDH1) mutation or an isocitrate dehydrogenase 2 (IDH2) mutation. In some embodiments, the methylation status is determined using reduced representation bisulfite sequencing (RRBS). In some embodiments, the subject is mammalian, preferably human.

In some embodiments, the present invention is directed to methods of detecting the presence of an isocitrate dehydrogenase mutation in a sample obtained from a subject which comprise measuring the amount of RBP1 messenger RNA and/or the amount of CRBP1 protein and/or the amount of retinoic acid in the sample. If the measured amount is less than that which is considered normal for WT samples, the presence of the mutation is indicated. If the mutation is indicated, the subject may be treated with one or more retinoids. In some embodiments, the isocitrate dehydrogenase mutation is an isocitrate dehydrogenase 1 (IDH1) mutation or an isocitrate dehydrogenase 2 (IDH2) mutation. In some embodiments, the subject is mammalian, preferably human.

In some embodiments, the present invention is directed to methods of providing a diagnosis and/or prognosis to a subject having a cancer, which comprises giving the diagnosis and/or prognosis to the subject based on the presence or absence of an isocitrate dehydrogenase mutation and/or the methylation status of all or part of the retinol-binding protein 1 (RBP1) promoter in a sample obtained from the subject, wherein the diagnosis is that the cancer may likely be treated with one or more retinoids where the isocitrate dehydrogenase mutation is present and/or the all or part of the RBP1 promoter is hypermethylated, and wherein the prognosis is that the subject will have an estimated time of survival that will likely be increased with treatment with one or more retinoids where the isocitrate dehydrogenase mutation is present and/or the all or part of the RBP1 promoter is hypermethylated. In some embodiments, the present invention is directed to methods of treating a subject having a cancer having been determined to be associated with an isocitrate dehydrogenase mutation and/or associated with RBP1 promoter hypermethylation, which comprises administering to the subject one or more retinoids. In some embodiments, the present invention is directed to methods of treating a subject having a cancer which comprises detecting the presence of an isocitrate dehydrogenase mutation in a sample obtained from the subject which comprises determining the methylation status of all or part of the retinol-binding protein 1 (RBP1) promoter in the sample, wherein the presence of the isocitrate dehydrogenase mutation is indicated where the methylation status of the all or part of the RBP1 promoter is determined to be hypermethylated; and if the isocitrate dehydrogenase mutation is present, administering one or more retinoids to the subject. In some embodiments, the present invention is directed to methods of treating a subject having a cancer which comprises sending a sample from the subject to another party to detect the presence of an isocitrate dehydrogenase mutation in the sample by determining the methylation status of all or part of the retinol-binding protein 1 (RBP1) promoter in the sample, wherein the presence of the isocitrate dehydrogenase mutation is indicated where the methylation status of the all or part of the RBP1 promoter is determined to be hypermethylated; receiving the results from the other party; and if the isocitrate dehydrogenase mutation is present, administering one or more retinoids to the subject. In some embodiments, the presence or association with the isocitrate dehydrogenase mutation and/or the methylation status of the all or part of the RBP1 promoter is or was determined using an assay according to the present invention, e.g. one of the assays as described in the above paragraphs and/or the detailed description below. In some embodiments, the subject is mammalian, preferably human.

In some embodiments, the present invention is directed to kits which comprise one or more reagents for conducting the assays as disclose herein, packaged together with a control or standard for comparison with the methylation profile obtained from a given subject and characterization. In some embodiments, the kits comprise one or more reagents for determining the methylation status of all or part of the retinol-binding protein 1 (RBP1) promoter in the sample from a subject packaged together with a standard or a control sample for characterizing the methylation status as being normal or hypermethylated. Such reagents may include (a) a bisulfite that converts the unmethylated cytosines to uracils, (b) a restriction enzyme that selectively cleaves the unmethylated cytosines, (c) a label specific for the unmethylated cytosines or the methylated cytosines, and/or (d) PCR reagents. In some embodiments, the control sample is/are nucleic acid molecule(s) to be assayed in parallel with the sample obtained from the subject. In some embodiments, the standard is a methylation profile of all or part of the wild type RBP1 promoter or a consensus methylation profile of the all or part of RBP1 promoters obtained from a plurality of normal subjects which corresponds to the all or part of the RBP1 promoter to be assayed with the kit. In some embodiments, the nucleic acid molecule or the all or part of the wild type RBP1 promoter or RBP1 promoters comprises, consists of, or consists essentially of 140740839-140741418 of chromosome 3. In some embodiments, the nucleic acid molecule or the all or part of the wild type RBP1 promoter or RBP1 promoters comprises, consists essentially of, or consists of CpG sites 5-25 of the CpG island located on chromosome 3 at 140740839-140741418. In some embodiments, the nucleic acid molecule or the all or part of the wild type RBP1 promoter or RBP1 promoters comprises, consists essentially of, or consists of one or more of the CpG sites of the RBP1 promoter. In some embodiments, the nucleic acid molecule or the all or part of the wild type RBP1 promoter or RBP1 promoters comprises, consists essentially of, or consists of at least 3, preferably at least 5, more preferably at least 10 CpG sites selected from the group consisting of CpG sites 14-24, 26-31, 39-45 and 59 of the CpG island located on chromosome 3 at 140740839-140741418. In some embodiments, the nucleic acid molecule or the all or part of the wild type RBP1 promoter or RBP1 promoters comprises, consists essentially of, or consists of at least 6, preferably at least 10, more preferably at least 15 CpG sites selected from the group consisting of CpG sites 1-5, 14-24, 26-31, 39-45 and 59 of the CpG island located on chromosome 3 at 140740839-140741418. In some embodiments, the nucleic acid molecule or the all or part of the wild type RBP1 promoter or RBP1 promoters comprises, consists essentially of, or consists of at least 10, preferably at least 15, more preferably at least 20 CpG sites selected from the group consisting of CpG sites 1-5, 14-24, 26-31, 39-45 and 59-62 of the CpG island located on chromosome 3 at 140740839-140741418. In some embodiments, the nucleic acid molecule or the all or part of the wild type RBP1 promoter or RBP1 promoters comprises, consists essentially of, or consists of one or more of the CpG sites 15, 21, 24, 29, 30, 40, 44 or 45 of the CpG island located on chromosome 3 at 140740839-140741418.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 shows a flow diagram of patient cohorts included in the analysis. The different patient cohorts and number of patients in each cohort are given. BiSEQ=bisulfite sequencing; GBM=glioblastoma multiforme; MSRE=methylation sensitive restriction enzyme; RBP1=retinol binding protein 1; RRBS=reduced representation bisulfite sequencing; TCGA=The Cancer Genome Atlas.

FIG. 2 illustrates the reduced representation bisulfite sequencing data processing protocol that was used to ensure high quality base calls and reads and consistency with the empirical sequencing protocol. Panel A) Sequencing reads which did not align to Human Genome NCBI Build 36 (HG18) were discarded. Panel B) Base pairs of reads with low base quality scores (PHRED<36) were truncated. Panel C) Reads with low alignment quality scores (MAPping quality (MAPA) score of less than 1.0), Panel D) misaligned reads that did not align to CCGG start sites, and Panel E) reads that did not map to MspI fragments less than 400 base pairs in length were discarded. Panel F) Base pairs for which the 3-prime end of the read extended beyond a CCGG site were truncated. A color version of this figure (as “Supplementary FIG. 1”) is available at HyperTextTransferProtocol://jnci.oxfordjournalsDOTorg/content/supp1/2012/07/30/dj s357.DC1/jnci_JNCI_{—}11_{—}1800_s01DOTpdf, where “HyperTextTransferProtocol” is “http” and “DOT” is “.”.

FIGS. 3A-3D show the RRBS in U87MG cells. FIG. 3A shows the percent coverage of CpG sites listed by each chromosome in U87MG cells. There were no major gaps in coverage on a chromosomal basis. FIG. 3B shows the average level of methylation in U87MG cells listed by each chromosome. FIG. 3C shows high levels of methylation in U87MG cells treated with CpG methyltransferease SssI served as the positive control, whereas FIG. 3D shows low levels of methylation in polymerase chain reaction-amplified genomic DNA from U87MG cells served as the negative control. Color versions of these figures (as “Supplementary FIGS. 2A-2D”) are available at HyperTextTransferProtocol://jnci.oxfordjournalsDOTorg/content/suppl/2012/07/30/dj s357.DC1/jnci_JNCI_{—}11_{—}1800_s01DOTpdf, where “HyperTextTransferProtocol” is “http” and “DOT” is “.” in which the chromosome ideograms show centromeres in red and patterns of Giemsa staining in different shades of grey.

FIGS. 4A-4C show the characterization of genome-wide differential methylation. FIG. 4A shows the hierarchical clustering of differentially methylated CpG islands (P<0.05, unpaired t-test) identified for IDH1 mutant (MUT) and wild-type (WT) tumors using RRBS. A color version of FIG. 4A is available in Chou et al. JNCI J Natl Cancer Inst (2012) 104(19): 1458-1469, which is herein incorporated by reference in its entirety. FIG. 4B provides a list of statistically significantly enriched annotation terms (enrichment score >1.30) associated with differentially methylated genes by DAVID Annotation Analysis. Terms with multiple enrichment scores denote multiple statistically significant clusters of genes with the specified annotation term. FIG. 4C shows the candidate hypermethylated genes in IDH1 MUT tumors. Statistically significantly methylated genes in a given dataset denotes an adjusted Q value of less than 0.05 (calculated by storey multiple comparison adjustment, unpaired ttest). MSRE=methylation sensitive restriction enzyme; NA=not available; RBP1=retinol binding protein 1; TCGA=The Cancer Genome Atlas.

FIGS. 5A-5B show RBP1 methylation and gene expression. FIG. 5A shows the CpG island methylation pattern for the RBP1 promoter. Sixty-two CpG sites were included in the analysis. IDH1 mutant (MUT) and wild-type (WT) status for the glioma samples was determined by reduced representation bisulfite sequencing. Solid arrows flank the location sequenced by bisulfite sequencing with the sequencing primer pair 1. FIG. 5B shows the relationship between RBP1 promoter methylation and gene expression using the Pearson correlation coefficient (R²) and the slope of the regression line ((3) using data from The Cancer Genome Atlas. The two-sided P value was based on the T statistic with 140 degrees of freedom. The black circles represent WT samples and grey squares represent IDH1 MUT samples. A color version of FIG. 5B is available in Chou (2012). All tumors from The Cancer Genome Atlas were glioblastoma multiforme (GBM, grade IV). MSRE=methylation sensitive restriction enzyme; A3=anaplastic astrocytoma (grade III); 03=anaplastic oligodendroglioma (grade III).

FIGS. 6A-6B show the methylation of RBP1. FIG. 6A is a histogram of methylation of retinol binding protein 1 determined by targeted bisulfite sequencing. Data from 198 patients was analyzed by IDH1/IDH2 mutation status. MUT=mutant; WT=wild-type. From left to right, the first and third bars are WT and the remaining bars are MUT. FIG. 6B provides the data of FIG. 6A in a table. Of the 198 patients: 29 had grade II gliomas, 32 had grade III gliomas, and 137 had grade IV gliomas. Methylation above 50% were classified as methylated. All methylated patients had >75% methylation. All unmethylated patients had <15% methylation. Difference in number of methylated patients between WT vs. IDH1/IHD2 MUT is statistically significant, P<0.001, two-sided Fisher's exact test. IDH1/IDH2 mutation strongly correlated with RBP1 methylation: P<0.001, Chi-Squared test.

FIGS. 7A-7B show the correlation between methylation detection methods. FIG. 7A shows the correlation between the percent methylation of RBP1 determined by RRBS with that determined by BiSEQ (n=10). FIG. 7B shows the correlation between the percent methylation of RBP1 determined by methylation sensitive restriction enzyme assay (MSRE) with that determined by BiSEQ (n=31). Spearman correlation test was done to determine R² and two-sided P values.

FIGS. 8A-8C shows RBP1 gene expression in human cell lines and glioma tumor samples. FIG. 8A shows RBP1 mRNA levels in the astrocyte progenitor cells (APC), human oligodendroglioma (HOG), U373, U138, and D54 cell lines as determined by quantitative real time reverse transcriptase polymerase chain reaction using β-actin as an internal control and standardized to the mRNA level for APC cells which was set as 100%. Data represent the mean and 95% confidence interval (whisker bars) from three independent experiments done in triplicate. FIG. 8B shows RBP1 mRNA levels in 56 samples from the Total Cohort (FIG. 1) were measured by quantitative real time reverse transcriptase polymerase chain reaction using β-actin as an internal control and standardized to the expression observed for normal brain cDNA (n=7). Data represent the mean from three independent experiments done in duplicate. FIG. 8C shows representative Western blots for CRBP1 expression in six cell lines, five normal brain samples, and eight tumor samples. Wild-type (WT) tumors (T1-T8) and IDH1/IDH2 mutant (MUT) tumors (T9-T16) were analyzed and α-tubulin was used as a loading control. The goat anti-CRBP1 antibody used for the cell lines produced two non-specific bands which are shown in the top panel of some samples. These non-specific bands were not observed with the rabbit anti-CRBP1 antibody used in the normal brain and tumor tissues.

FIG. 9A-9B show the Kaplan-Meier analysis of overall survival (OS) in a cohort of 124 primary glioblastoma multiforme patients using Cox proportional hazards analysis. FIG. 9A shows survival among patients with IDH1/IDH2 MUT (solid line, n=23) and WT (dashed, n=101) tumors. FIG. 9C shows survival among RBP1-methylated (solid line, n=22) and its RBP1-unmethylated (dashed line, n=102) patients. The number of patients at risk is given below each Kaplan-Meier curve. M=methylated; MUT=mutant; U=unmethylated; WT=wild-type.

FIGS. 10A-10B show the Kaplan-Meier analysis of overall survival (OS) in a cohort of 124 primary glioblastoma mulitforme patients with respect to RBP1 methylation and isotretinoin (ITR) treatment. FIG. 10A provides RBP1-unmethylated patients treated (solid, n=19) vs untreated with isotretinoin (dotted, n=83). FIG. 10B provides RBP1-methylated patients treated (solid, n=7) and untreated with isotretinoin (dotted, n=15). RBP1=retinol binding protein 1; WT=wild-type; MUT=mutant; ITR=isotretinoin.

FIG. 11 graphically shows the increased overall survival in patients with IDH1 mutant glioblastoma treated with isotretinoin. Survival for an unpublished preliminary cohort of 414 patients with primary glioblastoma were analyzed with Kaplan-Meier analysis. Survival curves for patients with wild-type or IDH1/2 mutant genotypes with or without upfront treatment with isotretinoin, in combination with standard-of-care debulking surgery, temozolomide, and radiation therapy, are shown. Upfront isotretinoin treatment is associated with only modest increase in overall survival for patients with wild-type tumors (WT). However, for patients with IDH1 or IDH2 mutant glioblastoma (IDH1 MUT), upfront treatment with isotretinoin was associated with significantly increased overall survival. WT=wild-type, MUT=IDH1 MUT, ITR=isotretinoin (13-cis retinoic acid).

### DETAILED DESCRIPTION OF THE INVENTION

The present invention is based on the unexpected discovery that hypermethylation in the retinol-binding protein 1 (RBP1) gene promoter is found in nearly all gliomas having mutations in isocitrate dehydrogenase 1 (IDH1) or isocitrate dehydrogenase 2 (IDH2) (collectively referred to as “IDH1/IDH2 mutant gliomas”) and is associated with improved patient survival and that disregulation of retinoic acid metabolism may contribute to glioma formation along the IDH1/IDH2-mutant pathway. RBP1 is located on 3q23 (UCSC Human Genome Browser HG18 chr3:140718972-140741180) and the sequence is publicly available.

RBP1 promoter hypermethylation in IDH1/IDH2 mutant gliomas was unexpectedly discovered when characterizing the CpG island methylation pattern in IDH1 mutant gliomas at high resolution by performing genome-wide RRBS on five pairs of pathologically matched wild-type and IDH1 MUT glioma tumor samples. As used herein, “IDH1/IDH2 MUT”, “IDH1/IDH2 mutation” and “MUT” are used interchangeably to refer to either an IDH1 or an IDH2 mutation except where the particular mutation is specifically identified. As used herein, “wild-type” or “WT” are used interchangeably to refer to no mutations in IDH1 or IDH2. Thus, for example, a “WT tumor” does not have any mutation in IDH1 or IDH2.

Hypermethylated promoter-associated CpG islands were identified in IDH1 MUT glioma samples, including the RBR1 promoter located on 3q23 (chr3: 140740839-140741418), which encodes the cytosolic retinol binding protein 1 (CRBP1) and is required for the efficient synthesis of all-trans retinoic acid (ATRA). Given the role of ATRA as an important transcription regulator, and the possibility that decreased CRBP1 activity may lead to alterations in ATRA metabolism and consequent transcription dysregulation, targeted bisulfite sequencing (BiSEQ) was performed to further validate RBP1 promoter hypermethylation.

Mutations in IDH1 and associated CpG island hypermethylation represent early events in the development of low grade gliomas and secondary glioblastomas. To identify candidate tumor suppressor genes whose promoter methylation may contribute to gliomagenesis, the methylation profiles of IDH1 MUT and WT tumors were compared using massively-parallel reduced representation bisulfite sequencing (RRBS).

As set forth in the experiments below RRBS was performed on ten pathologically-matched WT and MUT glioma samples and compared with data from a methylation-sensitive restriction enzyme technique and data from The Cancer Genome Atlas (TCGA). RRBS is a cost-effective technique for high-resolution methylome sequencing which uses restriction enzymes that cleave genomic DNA into fragments enriched for CpG sites (Meissner A, Nucleic Acids Res 2005; 33(18):5868-77; Meissner A, et al. Nature 2008; 454(7205):766-70). Methylation in RBR1 was identified in IDH1 mutant tumors and further analyzed with primer-based bisulfite sequencing. Correlation between IDH1/IDH2 mutation status and RBR1 methylation were evaluated with Spearman correlation. Survival data was collected retrospectively and analyzed with Kaplan-Meier and Cox proportional hazards analysis. All statistical tests were two-sided.

Methylome analysis identified coordinated CpG island hypermethylation in IDH1 MUT gliomas, consistent with previous reports. However, RBP1, important in retinoic acid metabolism, was unexpectedly found to be hypermethylated in 76 of 79 IDH1 MUT, 3 of 3 IDH2 MUT, and 0 of 116 WT tumors. IDH1/IDH2 mutation was highly correlated with RBR1 hypermethylation (n=198; Spearman R=0.94, 95% confidence interval=0.92 to 0.95, P<0.001). TCGA showed IDH1 MUT tumors (n=23) to be RBP1-hypermethylated with decreased RBP1 expression compared with WT tumors (n=124). Among patients with primary glioblastoma, patients with RBP1-unmethylated tumors (n=102) had decreased median overall survival compared with patients with RBP1-methylated tumors (n=22) (20.3 months vs 36.8 months, respectively; hazard ratio of death=2.48, 95% confidence interval=1.30 to 4.75, P=0.006).

### EXPERIMENTS

### Patient Cohorts and Tumor Specimens

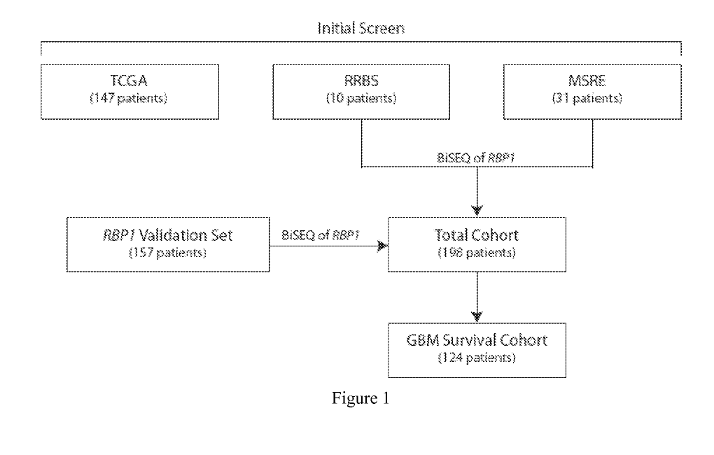

A total of 198 frozen or formalin-fixed paraffin embedded tissue specimens were obtained from the University of California, Los Angeles Brain Tumor Translational Resource (Los Angeles, Calif.). Remnant human brain tumor samples were collected from patients undergoing surgical resection and who provided written informed consent. The collection of human brain tumor samples was approved by the University of California, Los Angeles Institutional Review Board. Normal brain tissues were collected with University of California, Los Angeles Internal Review Board approval from one patient undergoing non-tumor related surgery and from four patients at the time of autopsy (the post-mortem interval was less than 12 hours). IDH1 was sequenced on all samples and IDH2 was sequenced on selected WT samples. A diagram showing the composition of the cohorts is shown in FIG. 1. The clinical characteristics of the patient cohorts are listed in Table 1 and Table 2 as follows:

TABLE 1
Characteristics of samples used in the initial methylation screen

| Characteristic | RRBS | MSRE* | TCGA |
|---|---|---|---|
| No. of patients | 10 | 31 | 147 |
| Mean age at diagnosis, | 45.7 (33-63) | 44.6 (21-75) | 55.2 (19-86) |
| y (range) | | | |
| Sex‡ | | | |
| No. of men | 6 | 16 | 87 |
| No. of women | 4 | 15 | 52 |
| Tissue pathology, | | | |
| No. of patients | | | |
| Astrocytoma grade II | 0 | 6 | 0 |
| Oligodendroglioma grade II | 0 | 6 | 0 |
| Oligoastrocytoma grade II | 0 | 0 | 0 |
| Astrocytoma grade III | 4 | 2 | 0 |
| Oligodendroglioma grade III | 4 | 1 | 0 |
| Oligoastrocytoma grade III | 0 | 2 | 0 |
| Glioblastoma grade IV | 2 | 14 | 147 |
| IDH1 mutation status | | | |
| WT | 5 | 15 | 124 |
| MUT | 5 | 16 | 23 |
| Tissue treated with | 0 | 4 | NA |
| RT/chemotherapy | | | |
| Tissue treated with | 0 | 0 | NA |
| isotretinoin | | | |
| Pretreatment history | — | — | |
| Yes | — | — | 17 |
| No | — | — | 121 |
| NA | — | — | 9 |

*The initial methylation sensitive restriction enzyme (MSRE) screen included data from 64 patients; however, data on the retinol binding protein-1 was available for only 31 patients. The MSRE technique does not have coverage of every gene on every sample. Thus, only data from these 31 patients are presented here.
IDH = isocitrate dehydrogenase
MUT = mutant
NA = not available
RRBS = reduced representation bisulfite sequencing
RT = radiotherapy
TCGA = The Cancer Genome Atlas
WT = wild-type
‡Data on sex for TCGA was not available for eight patients

TABLE 2
Demographics and RBP1 methylation status* of grade II-IV glioma samples (n = 198)

| | Total cohort† by targeted | | | GBM Survival Cohort | | |
|---|---|---|---|---|---|---|
| | bisulfite sequencing | | | within the total cohort | | |
| Demographic | Total | Methylated | Unmethylated | Total | Methylated | Unmethylated |
| No. of patients | 198 | 79 | 119 | 124 | 22 | 102 |
| Mean age at diagnosis, y (range) | 48.5 (19-79) | 39.5 (19-75) | 54.4 (21-79) | 53.0 (24-79) | 43.7 (24-75) | 55.0 (31-79) |
| Sex | | | | | | |
| No. of males | 122 | 51 | 71 | 70 | 14 | 56 |
| No. of female | 76 | 28 | 48 | 54 | 8 | 46 |
| Tissue pathology, No. of patients | | | | | | |
| Astrocytoma grade II | 9 | 7 | 2 | 0 | 0 | 0 |
| Oligodendroglioma grade II | 16 | 16 | 0 | 0 | 0 | 0 |
| Oligoastrocytoma grade II | 4 | 4 | 0 | 0 | 0 | 0 |
| Astrocytoma grade III | 13 | 7 | 6 | 0 | 0 | 0 |
| Oligodendroglioma grade III | 9 | 7 | 2 | 0 | 0 | 0 |
| Oligoastrocytoma grade III | 10 | 9 | 1 | 0 | 0 | 0 |
| Glioblastoma grade IV | 137 | 29 | 108 | 124 | 22 | 102 |
| WT | 116 | 0 | 116 | 101 | 0 | 101 |
| IDH1 MUT | 79 | 76 | 3‡ | 21 | 20 | 1 |
| IDH2 MUT | 3 | 3 | 0 | 2 | 2 | 0 |
| Tissue treated with RT/chemo | 14 | 11 | 3 | 0 | 0 | 0 |
| Tissue treated with isotretinoin | 0 | 0 | 0 | 0 | 0 | 0 |
| Tissue received no treatment | 183 | 67 | 116 | 124 | 22 | 102 |
| Pretreatment data not available | 1 | 1 | 0 | 0 | 0 | 0 |

*RBP1 methylation status was assessed by bisulfite sequencing in all 198 patients. If the average methylation level was greater than 50%, the sample was classified as methylated.
†The Total Cohort includes 41 patients from the initial screening cohort.
Chemo = chemotherapy;
IDH = isocitrate dehydrogenase;
MUT = mutant;
RBP1 = retinol binding protein 1;
RT = radiotherapy;
WT = wild type.
‡All IDH1 MUT unmethylated patients were grade 4 gliomas.

### Initial Methylation Screen

For RRBS, frozen tumor samples consisting of 5-pairs of pathologically matched WT and IDH1 MUT World Health Organization grade III and IV glioma tumor samples were analyzed. Methylation data from 31 patients that had coverage of RBP1 was included for analysis by methylation sensitive restriction enzyme assay (MSRE). Data from a total of 64 patients were used in the initial methylation screen. Thirty-three of the 64 samples did not have coverage at RBP1 and were not considered further. MSRE data on 34 of 64 patients were previously reported (Lai A, et al. J Clin Oncol 2011; 29(34):4482-90). For The Cancer Genome Atlas (TCGA), IDH1 genotype, methylation, and gene-expression data were available on 147 grade IV tumor samples from the TCGA database (Noushmehr H, et al. Cancer Cell 2010; 17(5):510-22; Network TCGAR. Nature 2008; 455(7216):1061-8).

### Total Cohort to Assess RBP1 Methylation by Bisulfite Sequencing

RBP1 methylation was assessed in a total of 198 retrospectively identified frozen or formalin-fixed paraffin embedded samples by BiSEQ. The Total Cohort included 41 patients that were included in the initial methylation screen (10 from RRBS, 31 from MSRE) and a validation set of 157 patients. RBP1 methylation status for all 198 patients was assessed by BiSEQ.

### GBM Survival Cohort

Within the 198-patient Total Cohort, 124 samples that were obtained from treatment-naïve primary GBM patients with detailed clinical information were retrospectively identified. Patients were treated with a combination of radiation (RT) and temozolomide (TMZ) after surgical resection (Stupp R, et al. N Engl J Med 2005; 352(10):987-96). Overall survival (OS) was defined as the date of diagnosis to date of death from any cause. For patients lost to follow-up without obtainable date of death, censoring date was last clinic visit or contact. If the last clinic or contact was after the Sep. 9, 2011 freeze date, the patient was censored at Sep. 22, 2011. These patients were a part of two other studies examining clinical and molecular features of IDH1 mutant gliomas (Lai 2011) and the prognostic values of 06-methylguanine-DNA methyl transferase promoter methylation on patient survival (data not shown).

### Cell Culture

The U87MG, U138MG, U373MG glioma cell lines were obtained from Dr. Paul Mischel (University of California, Los Angeles, Los Angeles, Calif.). Dr. Carol Kruse (University of California, Los Angeles, Calif.) provided the D54MG cell line. Dr. Glyn Dawson (University of Chicago, Chicago, Ill.) and Dr. Anthony Campangoni (University of California, Los Angeles, Calif.) provided the UHOG cell line. All glioma cell lines were cultured in Dulbecco's Modified Eagle Medium/F12 Medium (Invitrogen, Grand Island, N.Y.) supplemented with 10% fetal bovine serum and 100 U/mL penicillin/streptomycin. The human astrocytic progenitor cell line (APC) was obtained from Dr. Ina Wanner (University of California, Los Angeles) and cultured in Dulbecco's Modified Eagle Medium/F12 medium with 10% fetal bovine serum (Wanner IB. Astrocytes: Methods and Protocols. In: Milner R, ed. Springer, New York: Humana Press Inc.; in press).

### Massively Parallel Reduced Representation Bisulfite Sequencing

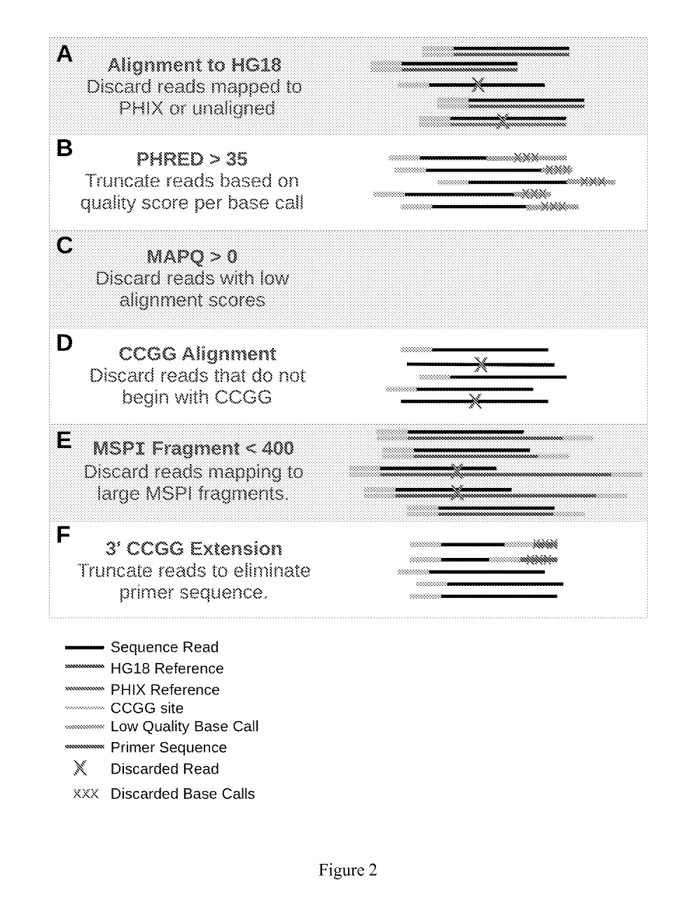

Reduced representation bisulfite sequencing was done using the protocol published by Meissner et al. (Meissner 2005; Meissner 2008). Details of the RRBS protocol, including generation of U87MG genomic DNA samples, RRBS quality control, and bioinformatics, are outlined in FIG. 2. Briefly, DNA was isolated from U87MG glioma cells and frozen tumor tissues, digested with the restriction enzyme MspI to enrich for fragments containing CpG islands, and end repaired using methylated cytosine. After adapter ligation, the DNA was size fractionated by gel electrophoresis, and DNA fragments between 100-400 base pairs in length were isolated to minimize large fragments with poor sequencing coverage. Isolated DNA were then bisulfite treated, amplified, mixed with unmodified PhiX DNA (a bacterial genome inserted for quality control and to assess mapping), and sequenced on an ILLUMINA GENOME ANALYZER IIx (San Diego, Calif.). The NOVOALIGN software package (Novocraft Technologies, Selangor, Malaysia) was used to align the sequence data. Aligned sequence data was then sorted using the SAMTooLs software package (Li H, et al. Bioinformatics 2009; 25(16):2078-9), and stored in SAM format for further analysis. Methylation status was determined at individual CpG sites, and the results were compiled to show the level of methylation at individual CpG islands. CpG islands were mapped by previously published definitions (Gardiner-Garden M, et al. J Mol Biol 1987; 196(2):261-82); and a strict quality control protocol was implemented to ensure the quality of the sequence base calls, alignment, and the final methylation data.

Specifically, genomic DNA was isolated from U87MG cells using the DNEASY BLOOD AND TISSUE KIT (Qiagen, Venlo, Netherlands). Fully methylated U87MG DNA was prepared by treating 4 μg genomic DNA from normal brain tissue with 16 units of SssI enzyme (NEB, Ipswich, Mass.) and 160 nM S-Adenosylmethione for 6 hours at 37° C. twice. The product was purified using the ZYMO CLEAN AND CONCENTRATOR KIT (Zymo Research Corp., Orange, Calif.), in which the DNA was eluted using 20 μL, of polymerase chain reaction (PCR)-grade H₂O. The typical yield after purification was 2 μg. Fully unmethylated U87MG DNA was generated by subjecting 10 ng of genomic DNA from normal brain tissue to whole-genome amplification with the GENOMIPHI V2 AMPLIFICATION KIT (Amersham Biosciences, Piscataway, N.J.) following the manufacturer's instructions with unmodified dCTP. The typical yield was about 4-7 μg.

Reduced representation bisulfite sequencing (RRBS) was done using the protocol published by Meissner et al. (Meissner 2005; Meissner 2008). Briefly, DNA was isolated from U87MG glioma cell lines and frozen tumor tissues, digested with the restriction enzyme MspI, and end repaired using methylated cytosine. After adapter ligation, the DNA was size fractionated by gel electrophoresis. DNA fragments between 100-400 base pairs in length were isolated, bisulfite treated, amplified, and mixed with Phix DNA. The resulting mixture contained 90% bisulfite-treated DNA and 10% Phix control DNA.

An ILLUMINA GENOME ANALYZER IIx (Illumina, San Diego, Calif.) was used to sequence the DNA as previously described (Clark M J, et al. PLoS Genet 2010; 6(1):e1000832). One hundred twenty qseq files were generated; each file contained reads that were 76 base pairs in length, with about 40 million reads per sample, which were aligned to both the human (HG18) and Phix genomes. The NOVOALIGN software package (Novoalign v2.08.01, Novocraft Technologies, Selangor, Malaysia), which includes functionality to align bisulfite treated DNA, was used to align the sequence data to both genomes. Aligned sequence data was then sorted using the SAMTOOLS software package (Li 2009), and stored in SAM format for further analysis.

In terms of quality control, about 40% of the total reads aligned to reference HG18, and 10% aligned to the Phix genome. Several steps were taken to identify and exclude low quality and/or poorly aligned reads as shown in FIG. 2. PHRED scores, assigned by the sequencer (obtained from the Phred software package, available from CodonCode Corporation, Centerville, Mass., see also WorldWideWeb.phrapDOTcom, where “WorldWideWeb” is “www” and “DOT” is “.”), were used to assess the quality of each base call within each aligned read. The scores ranged from 35 (low) to 73 (high). As expected, the number of low quality base calls increased towards the 3-prime end of each read. Despite this, the mean quality scores at each base position remained above 60. On the basis of the distribution of PHRED scores across all aligned reads, base calls with PHRED scores of less than 36 were excluded. MAPping Quality (MAPQ) scores, ranging from 0 to 150, were assigned by NOVOALIGN to quantify the mapping quality of each aligned read. The majority of aligned reads (97.7%) had high quality mapping scores. On the basis of the distribution of MAPQ scores, reads with scores equal to zero were excluded from further analysis. In addition to PHRED and MAPQ scores, size selection and restriction digest with MspI also allowed us to identify sequencing and alignment errors. Theoretically, because MspI cuts at CCGG sites, the 5′ end of each read should align to CCGG in the reference genome. In addition, because of size selection, the reads should neither map to MspI fragments (predicted in silico) greater than 400 bp, nor extend beyond a CCGG site at the 3′ end. It was observed that 7% of reads did not have a 5′ CCGG start site and 1% of reads mapped to predicted fragments larger than 400 bp. Low MAPQ scores for these reads confirmed misalignment, so they were excluded from analysis. About 10% of reads showed 3′ extension beyond a CCGG site. Although these reads mapped to predicted fragments smaller than expected, MAPQ scores did not indicate misalignment. Thus, the reads were truncated at the 3′ CCGG site and kept for further analysis.

For CpG island methylation scoring, methylation was first determined by comparing each CG site within a read to the reference sequence. For forward strand alignment, a C-T mismatch at the C position of a CG site indicated bisulfite conversion, whereas a C-C match indicated methylation. Similarly, for reverse strand alignment, a G-A mismatch at the G position of a CG site indicated bisulfite conversion, and a G-G match indicated methylation. Using these designations, the number of reads and the percent methylation was summed for each CG site. Overall methylation scores were then assigned to each known CpG island (Rhead B, et al. Nucleic Acids Research 2010; 38:D613-D619; Karolchik D, et al. Nucleic Acids Research 2004; 32:D493-D496; Gardiner-Garden 1987) by calculating the average methylation across all CG dinucleotides within an island, weighted by the coverage at each CG site.

### Methylation Sensitive Restriction Enzyme Assay

A methylation sensitive restriction enzyme assay (MSRE), as described by Tran et al. (Tran A, et al. Front Neurosci 2009; 3:57), was used to assess genome wide methylation for 64 glioma samples. Briefly, genomic DNA was digested with the restriction enzyme BfaI and divided into two aliquots. One aliquot was digested with the methylation-sensitive restriction enzyme HpaII and labeled with Cy5 after polymerase chain reaction (PCR)-amplification (Tran 2009). The second aliquot was digested with the methylation-insensitive enzyme MspI and labeled with Cy3 after PCR amplification. The PCR products were hybridized to an AGILENT HIGH-DENSITY 2-COLOR HUMAN CPG ISLAND MICROARRAY (Agilent Technologies, Santa Clara, Calif.).

In silico digestion of the whole human genome (HG18) using enzyme BfaI was used to predict sensitive (containing CCGG) and insensitive (not containing CCGG) fragments of DNA from tumor samples. Each BfaI fragment was assigned a methylation score based on the median Loess corrected log ratios of the Cy-5/Cy-3 signal for all probes mapping to that fragment. Methylation scores were then standardized on the basis of the distribution of values for all insensitive fragments.

### Analysis of Methylation and Gene-Expression in The Cancer Genome Atlas Dataset

Methylation data for 147 glioblastoma samples was obtained from the The Cancer Genome Atlas (TCGA) (Noushmehr 2010). Methylation was measured at about 27,000 CpG dinucleotides for 62 samples, using the ILLUMINA INFINIUM HUMAN METHYLATION 27 ASSAY (Illumina, San Diego, Calif.), and at 1500 CpG dinucleotides for 85 samples, using the ILLUMINA GOLDENGATE METHYLATION CANCER PANEL ASSAY (Illumina, San Diego, Calif.). Level 3 data, including normalized methylation signal per gene per sample, was downloaded directly from the TCGA data portal (HyperTextTransferProtocol://tcga-data.nci nihDOTgov/tcga/, where “HyperTextTransferProtocol” is “http” and “DOT” is “.”). Gene expression data was available for 142 of 147 TCGA samples. Gene expression was measured using the AFFYMETRIX HT HUMAN GENOME U133A MICROARRAY (Affymetrix, Santa Clara, Calif.). A TCGA gene-expression probe (203423_at, chr3:140718973-140741180) covers transcript variant 1 of the RBP1 gene.

### Statistical Analysis

Differentially methylated CpG islands located in gene promoter regions were identified by performing the student's t-test comparing IDH1 MUT and WT samples. To minimize the number of statistically significant but biologically non-significant changes in methylation, arbitrary thresholds of 0.4, 0.4, and 3 (for RRBS, TCGA, and MSRE, respectively) were set as the minimum difference between the mean levels of methylation between WT and IDH1 MUT samples that may be biologically significant to warrant further evaluation. Different cutoffs were chosen for the three different datasets because of their technical differences. Data for the MSRE technique is expressed as a log ratio whereas data for the RRBS technique is expressed as the mean difference. Also, to control the false discovery rate, a Q of less than or equal to 0.05 was set as the threshold for statistical significance, as described by Storey et al. (Journal of the Royal Statistical Society Series B-Statistical Methodology 2002; 64(3):479-498), for the three datasets. A false discovery rate of 0.02% was estimated via permutation testing of the RRBS data (252 permutations (Noy N. Biochem J2000; 348 Pt 3:481-95), HyperTextTransferProtocol://biosunl.harvardDOTedu/˜cli/complab/dchip/ or HyperTextTransferProtocol://sites.googleDOTcom/site/dchipsoft/, wherein “HyperTextTransferProtocol” is “http” and “DOT” is “.”).

Correlation between IDH1 mutation and RBP1 methylation was determined using the Spearman correlation test. To examine the relationship between overall survival (OS) and RBR1 methylation, survival curves were estimated by the Kaplan-Meier method and groups were compared using a Log-rank test. Because of the tight correlation between IDH1/IDH2 mutations and RBP1 methylation, multivariable analysis with Cox proportional hazards model was done and included variables including age (years, continuous variable), sex (male or female), performance status (Karnofsky Performance Score 80-100 vs≦70), extent of resection (gross-total vs sub-total/biopsy), and IDH1/IDH2 mutation status or RBP1 methylation status separately. Statistical analyses by race/ethnic group were not done. The assumption of proportionality was verified by the statistical test of correlation between Schoenfeld residuals and ranked survival time. Statistical analyses were performed using the open-source R statistical analysis package (available from HyperTextTransferProtocol://WorldWideWeb/R-projectDOTorg, wherein “HyperTextTransferProtocol” is “http”, “WorldWideWeb” is “www”, and “DOT” is “.”). All tests were two-sided and a P value of less than 0.05 was considered statistically significant.

### Targeted Bisulfite Sequencing of RBP1

The methylation status of the RBP1 promoter CpG island was assessed by standard bisulfite sequencing utilizing a nested PCR protocol with the primer sets:

stage 1, forward:
(SEQ ID NO: 1)
5′-TTTATTGGGTATTGGAAGATGTTG-3′
and
reverse:
(SEQ ID NO: 2)
5′-TCCAATCTACAACCTAAAAACTACC-3′,
and
stage 2, forward:
(SEQ ID NO: 3)
5′-GGTATTGGAAGATGTTGGTTAA-3′
and
same reverse primer as stage 1.

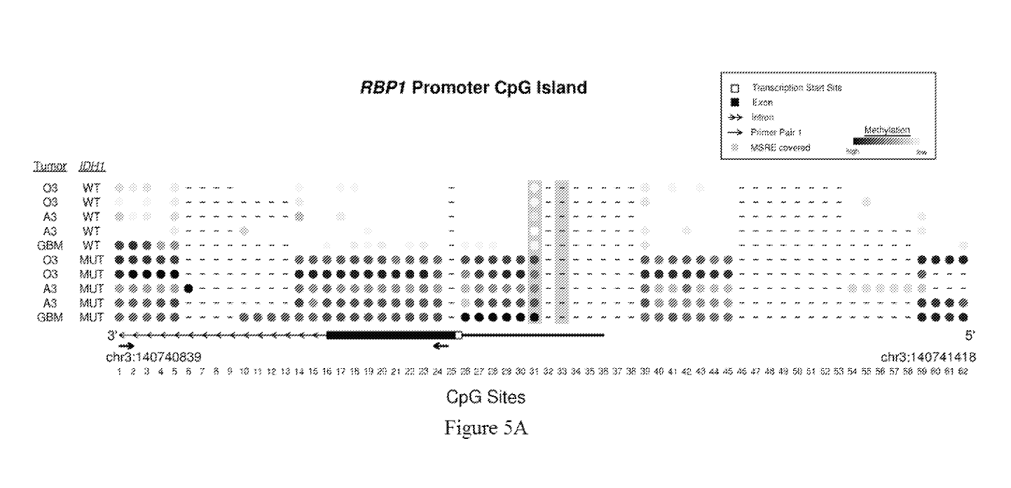

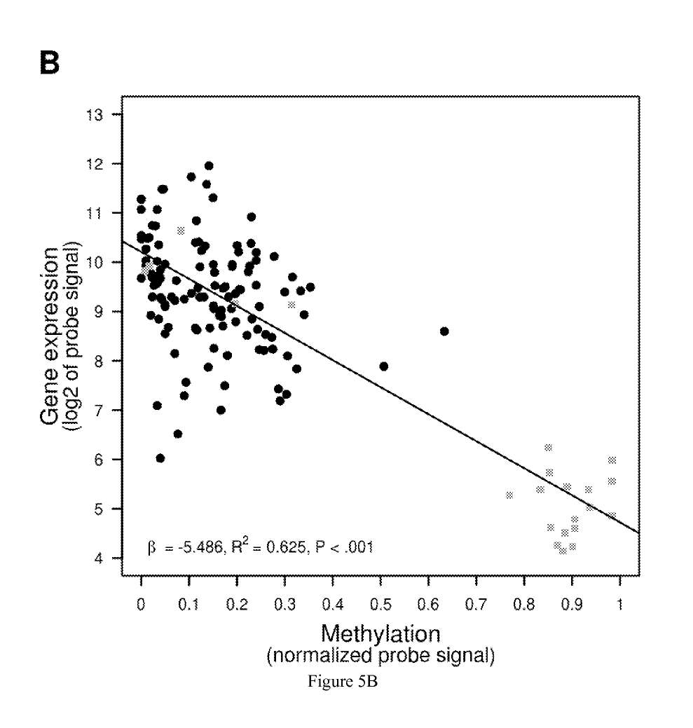

The sequence of each sample was determined using CHROMAS LITE 2.33 (Technelysium Pty Ltd, South Brisbane, QLD, Australia). The level of methylation was semi-quantitatively scored in quartiles by the relative heights of the methylated and unmethylated peaks. For RBP1 methylation determination by BiSEQ, the mean methylation levels (of the 21 CpG sites sequenced) above 50% were classified as methylated. Specifically, 21 CpG sites in the whole CpG island, corresponding to CpG sites 5 to 25 in FIG. 5A, were assessed for methylation individually, whether 0-100%, e.g. 0, 25, 50, 75, or 100%. Then all the methylation levels of CpG sites were averaged to give the indicated methylation percent which is deemed to most closely represent the methylation level of the given CpG island. Stage 2 may be omitted when DNA is isolated from high-quality tissue such as frozen tumor tissue. The nested 2-stage protocol may be used for DNA isolated from paraffin-embedded tissue.

### IDH1 and IDH2 Sequencing

Genomic DNA was isolated from formalin-fixed paraffin embedded or frozen tissue using the RECOVERALL TOTAL NUCLEIC ACID ISOLATION KIT (Invitrogen, Grand Island, N.Y.). Sequencing of IDH1 at residue 132 and IDH2 at residue 172 was determined by Sanger sequencing with the following primers:

IDH1 forward:
(SEQ ID NO: 4)
5′-GCGTCAAATGTGCCACTATC-3′
and
reverse:
(SEQ ID NO: 5)
5′-GCAAAATCACATTATTGCCAAC-3′;
and
IDH2 forward:
(SEQ ID NO: 6)
5′-CTCACAGAGTTCAAGCTGAAG-3′
and
reverse:
(SEQ ID NO: 7)
5′-CTGTGGCCTTGTACTGCAGAG-3′.

Purified PCR products were sequenced using the BIGDYE TERMINATOR v1.1 (Applied Biosystems, brand of Life Technologies, Carlsbad, Calif.) and analyzed on a 3730 sequencer (Applied Biosystems).

### Analysis by Quantitative Real-Time Reverse Transcriptase PCR

Total RNA was extracted from culture cells or tumor tissues using Trizol (Invitrogen, Grand Island, N.Y.) according to the manufacturer's instructions. Purity of the total RNA was then determined by the 260/280 nm ratio and the integrity was checked by electrophoresis on 1% agarose gel.

One microgram of RNA from each sample was reverse transcribed to cDNA with the Reverse Transcription System (Promega, San Luis Obispo, Calif.) using oligo-dT primers. Normal brain cDNA isolated from one frozen surgical tissue, four frozen autopsy tissue, and two commercially available cDNA libraries (Biochain, Hayward, Calif.; Invitrogen, Grand Island, N.Y.) were used as controls. Reverse transcriptase PCR (RT-PCR) was performed using PLATINUM DNA POLYMERASE (Invitrogen, Grand Island, N.Y.). The PCR product was separated on a 3% agarose gel. Quantitative real-time RT-PCR (qRT-PCR) using FASTSTART UNIVERSAL SYBRR-GREEN MASTER (Roche, Mannheim, Germany) using a LIGHTCYCLER 480 System (Roche, Mannheim, Germany) was done. The primers were designed with PRIMER3 (Rozen, 2000 #1466) (version 0.4.0, available from HyperTextTransferProtocol://frodo.wi.mitDOTedu, wherein “HyperTextTransferProtocol” is “http” and “DOT” is “.”) software and were as follows:

forward:
(SEQ ID NO: 8)
5′-CAACTGGCTCCAGTCACTCC-3′
and
reverse:
(SEQ ID NO: 9)
5′-TGCACGATCTCTTTGTCTGG-3′.

The following conditions were used for amplification: 95° C. for 3 minutes, 40 cycles at 95° C. for 10 seconds, followed by 60° C. for 30 seconds, and 72° C. for 30 seconds. All samples were amplified in duplicate from the same RNA preparation and the results are presented as the mean with standard error of the mean from three independent experiments. RBP1 mRNA levels were standardized to the levels of β-actin (internal control) and quantified by the relative Ct method (2^{ΔΔCt}).

### Protein Expression by Western Blot

Total protein lysates were prepared using radio-immunoprecipitation assay buffer containing protease inhibitor to lyse the cells and tissues. The proteins (15 μg) were separated on a 4%-20% sodium dodecyl sulfate polyacrylamide gel and transferred to nitrocellulose membranes (0.45 μm, Bio-Rad, Hercules, Calif.). Western blot was performed with goat anti-CRBP1 (1:200) or rabbit anti-CRBP1 (1:200) polyclonal antibody (Sigma, St. Louis, Mo., USA), mouse anti-α-tubulin monoclonal antibody (1:4000, Sigma, St. Louis, Mo., USA), horseradish peroxidase-conjugated rabbit anti-goat (1:8000) or goat anti-rabbit (1:8000) IgG (Santa Cruz Biotech, Santa Cruz, Calif., USA), horseradish peroxidase-conjugated goat anti-mouse IgG (1:10000, Jackson ImmunoResearch, West Grove, Pa.) and an enhanced chemiluminescence detection kit (Pierce, Rockford, Ill.). Densitometry was performed with GEL-PRO ANALYZER 4.0 software (Media Cybernetics, Bethesda, Md.).

### Development of RRBS for Methylome Profiling in Glioma

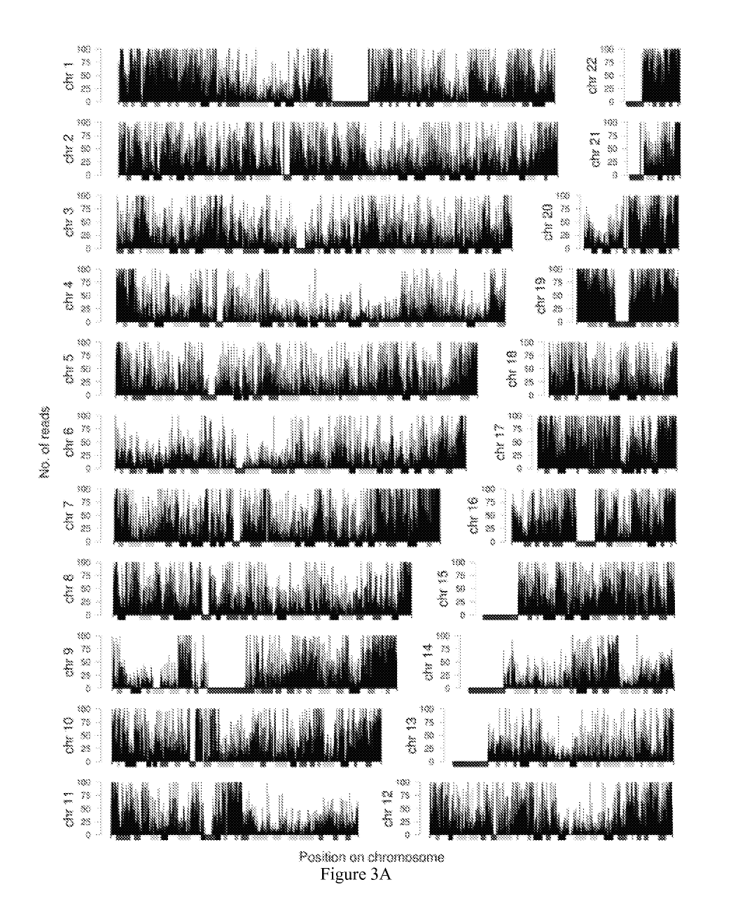

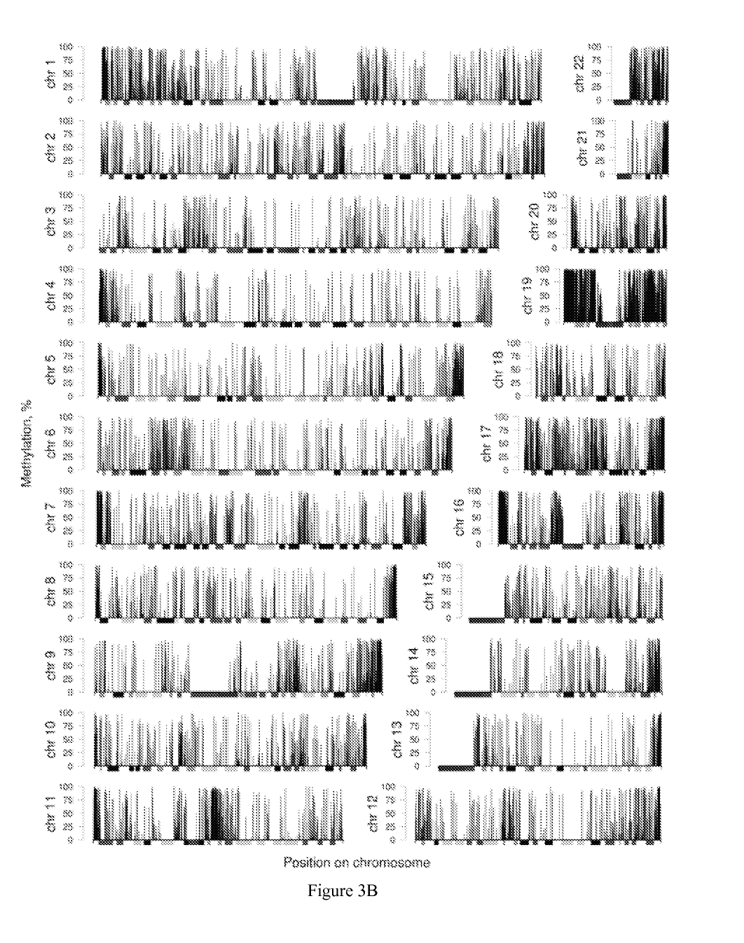

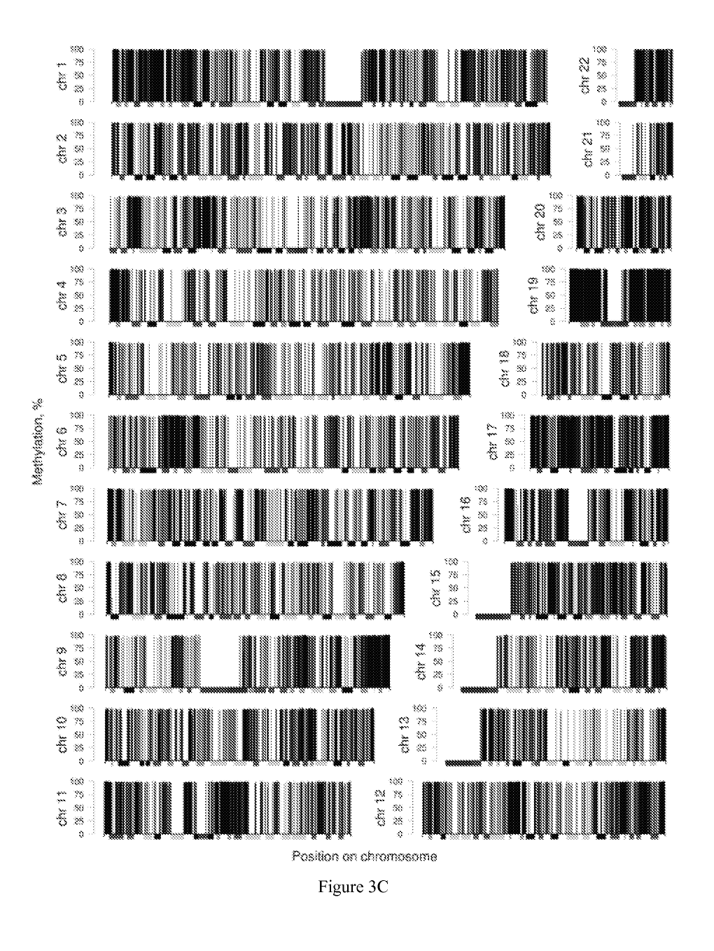

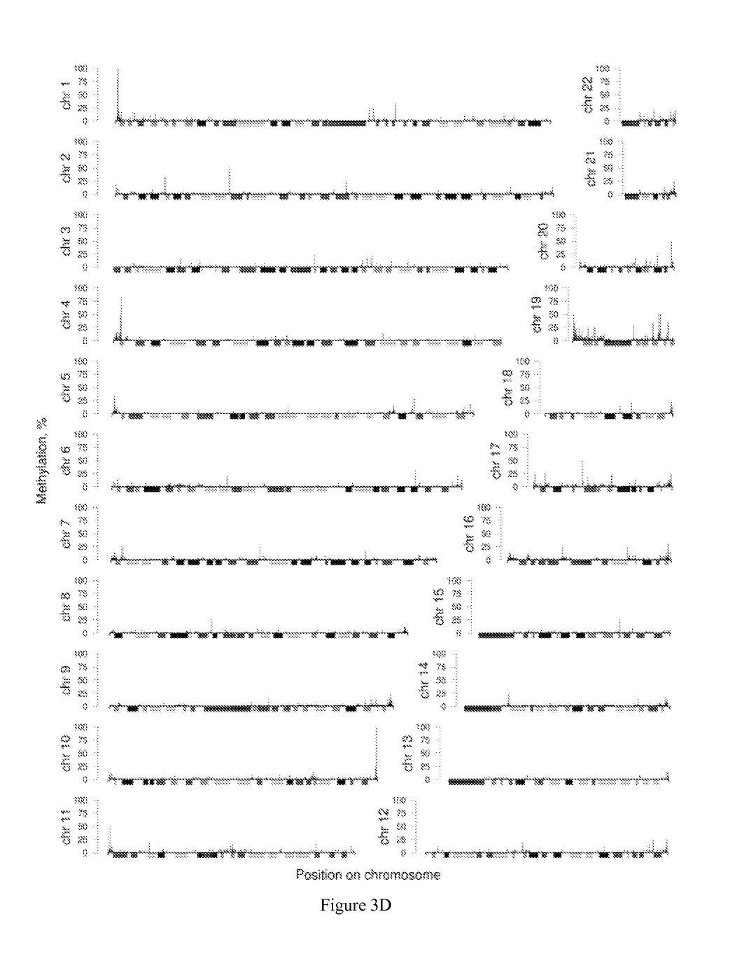

To examine potential tumor suppressor genes that may be hypermethylated in IDH1 mutant tumors, the RRBS protocol (Meissner 2005; Meissner 2008) was applied to profile the glioma methylome. As a part of the protocol validation, RRBS on DNA isolated from U87MG cells, a widely used glioblastoma cell line, was initially performed. U87MG DNA that had been treated with the CpG methyltransferase enzyme SssI was used as positive control. A negative control was obtained from PCR-amplified genomic DNA for which CpG methylation was not amplified. Data from U87MG DNA demonstrated no major gaps in the sequencing coverage (FIG. 3A) and Table 3, as follows, provides the expected methylation patterns in the positive and negative controls (see also FIGS. 3B-3D).

TABLE 3
Reduced representation bisulfite sequencing in U87MG cells and frozen glioma tissues

| | | U87MG | U87MG | WT | IDH1 MUT |
|---|---|---|---|---|---|
| | | negative | positive | mean | mean |
| Sample | U87MG | control | control | (n = 5) | (n = 5) |
| Total No. of reads | 34,406,353 | 78,527,855* | 121,683,196* | 38,279,367 | 38,344,371 |
| No. of aligned reads | 15,018,877 | 11,124,619 | 20,946,145 | 17,182,263 | 16,466,007 |
| No. of CpG sites covered | 2,141,620 | 1,233,669 | 2,094,504 | 2,293,879 | 2,083,096 |
| Average reads per CpG site | 19.01 | 17.81 | 24.11 | 20.16 | 20.93 |
| Total No. of CpG islands | 26,567 | 26,567 | 26,567 | 26,567 | 26,567 |
| No. of CpG islands covered | 23,540 | 22,102 | 23,254 | 23,985 | 23,923 |
| Coverage within island, % | 0.552 | 0.423 | 0.512 | 0.589 | 0.592 |
| Total No. of unique refseq genes | 21,338 | 21,338 | 21,338 | 21,338 | 21,338 |
| No. of unique refseq genes covered | 17,854 | 18,114 | 17,933 | 18,112 | 17,467 |
| Total No. of unique promoters | 21,338 | 21,338 | 21,338 | 21,338 | 21,338 |
| No. of unique promoters covered | 14,280 | 13,687 | 14,011 | 14,588 | 14,621 |
| Mean CpG island methylation†, (SEM) | 30.14 (0.23) | 1.12 (0.02) | 97.29 (0.02) | 28.26 (0.11)‡ | 32.80 (0.11) |
| Methylated CpG sites, % | 43.97 | 0.037 | 99.35 | 54.44 | 55.48 |

*These samples were analyzed using an Illumina HiSeq 2000 system (Illumina Inc., San Diego, CA).
IDH = isocitrate dehydrogenase;
MUT = mutant;
WT = wild type.
†Data represent the mean and standard of the mean (SEM).
For reference, the high-resolution methylome of U87MG derived from RRBS is listed in Supplementary Table 1 (available online at HyperTextTransferProtocol://jnci.oxfordjournalsDOTorg/content/suppl/2012/07/30/djs357.DC1/jnci_JNCI_11_1800_s03DOTxlsx, wherein “HyperTextTransferProtocol” is “http” and “DOT” is “.”.

### Methylated Genes in Wild-Type and IDH1 Mutant Gliomas

RRBS was then applied to characterize the methylome of five pairs of pathologically matched WT and IDH1 MUT frozen glioma samples. The clinical characteristics of the ten tumors are listed in Table 1. Similar to results with U87MG DNA, about 19 million aligned reads were generated per sample, yielding methylation information on about 2 million CpG sites within 23,900 CpG islands, with an average of 59% coverage per island (Table 3). About 14,000 covered CpG islands were associated with unique promoters.

The mean methylation levels of each CpG island were compared between the five WT and five IDH1 MUT tumors. Using an absolute difference in methylation greater than 40% and a cutoff of P less than or equal to 0.05, 346 CpG islands were found to be statistically significantly differentially methylated between WT and IDH1 MUT tumors; 125 of the CpG islands were associated with promoters (Supplementary Table 2, available online at HyperTextTransferProtocol://jnci.oxfordjournalsDOTorg/content/suppl/2012/07/30/dj s357.DC1/jnci_JNCI_{—}11_{—}1800_s20DOTxlsx, where “HyperTextTransferProtocol” is “http” and “DOT” is “.”. Given the small sample size, only 81 CpG islands, including RBP1, passed multiple testing correction with Q less than or equal to 0.05. The false discovery rate in this cohort, using the above statistical significance criteria with correction, was estimated at less than 0.02% by permutation testing.

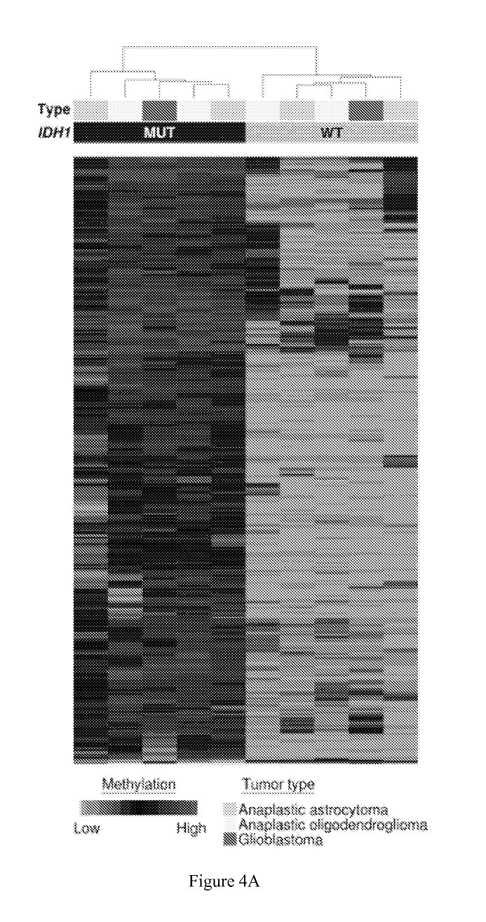

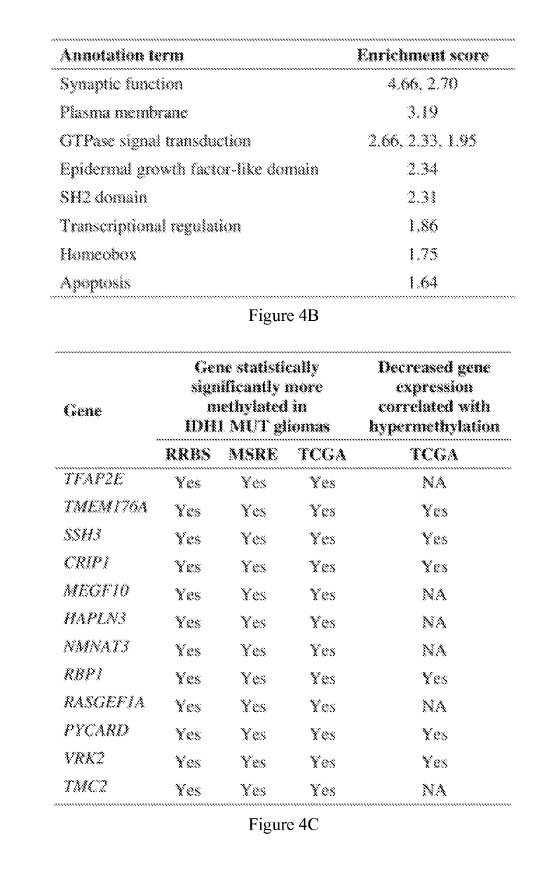

Whole genome characterization revealed that global methylation pattern of IDH1 MUT tumors was statistically significantly increased compared with WT tumors (Table 3 and FIG. 4A). This finding is consistent with a Glioma CpG Island Methylator Phenotype observed by others (Noushmehr 2010; Christensen 2011). Compared with publicly available data, 106 of the 346 genes identified in this study were among the 1550 hypermethylated genes found by Noushmehr et al. (Noushmehr 2010). However, the experiments and results herein are the first to identify the correlation between RBP1 hypermethylation and IDH1/IDH2 MUT gliomas.

### RBP1 Hypermethylation in IDH1 and IDH2 Mutant Gliomas

Annotation analysis of hypermethylated genes using the software package DAVID (Huang da W, et al. Nat Protoc 2009; 4(1):44-57) revealed enrichment of several annotation terms including transcriptional regulation and apoptosis (FIG. 4B). To screen for candidate tumor suppressor genes that were hypermethylated in IDH1 mutant tumors, employing criteria that the hypermethylated CpG island had to be in the 5′ promoter region, show statistically significant hypermethylation within all three screening datasets (RRBS, MSRE, and TCGA), and correlate with decreased gene expression from the TCGA dataset. Several genes met these screening criteria and a portion of the complete list (346 hypermethylated CpG islands, Supplementary Table 2) is provided in FIG. 4C. As set forth in Table 4, one of the identified genes was RBP1 (FIG. 5A and Table 4).

TABLE 4
RBP1 methylation by reduced representation bisulfite sequencing (RRBS), methylation
sensitive restriction enzyme (MSRE), and The Cancer Genome Atlas (TCGA) analysis*

| | | WT | | IDH1 MUT | | | | | |
|---|---|---|---|---|---|---|---|---|---|
| | | | Mean | | Mean | | | | |
| | No. of | No. of | methylation | No. of | methylation | Possible | | | |
| Assay | patients | patients | (SD) | patients | (SD) | range | Actual range | P† | P‡ |
| RRBS | 10 | 5 | 0.05 (0.02) | 5 | 0.68 (0.06) | 0-1.0 | 0.01-0.81 | <.001 | .008 |
| MSRE | 31 | 15 | 4.05 (0.57) | 16 | 7.82 (0.49) | −9.19-21.75 | 1.68-10.95 | <.001 | <.001 |
| TCGA | 147 | 124 | 0.14 (0.01) | 23 | 0.69 (0.08) | 0-1.0 | 0-0.98 | <.001 | <.001 |

*For RRBS and TCGA, the mean methylation represents the percentage of methylation in the region of interest. For MSRE, mean methylation represents standard deviations above known baseline (unmethylated) signal.
IDH = isocitrate dehydrogenase;
MUT = mutant;
RBP1 = retinol binding protein 1;
WT = wild type
†Two-sided P was calculated by Student t test.
‡Two-sided P was calculated by Wilcoxon test.

Analysis of data obtained from the TCGA database showed increased RBP1 methylation in 23 IDH1 mutant tumors compared with 124 WT tumors (FIG. 5B, and Table 4), and RBP1 promoter methylation was associated with decreased expression of CRBP1 in 141 GBM samples from the TCGA (R²=0.625, P<0.001) (FIG. 5B).

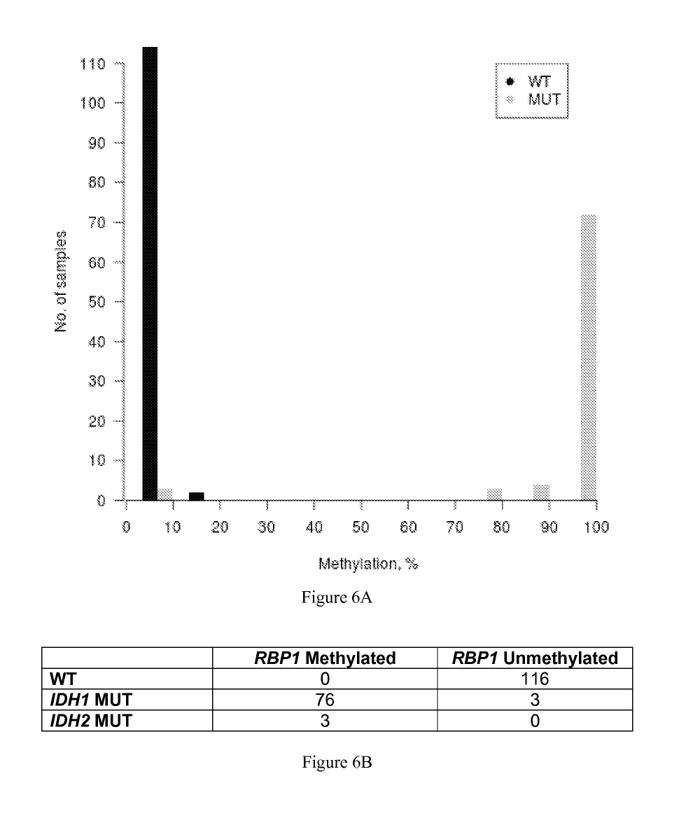

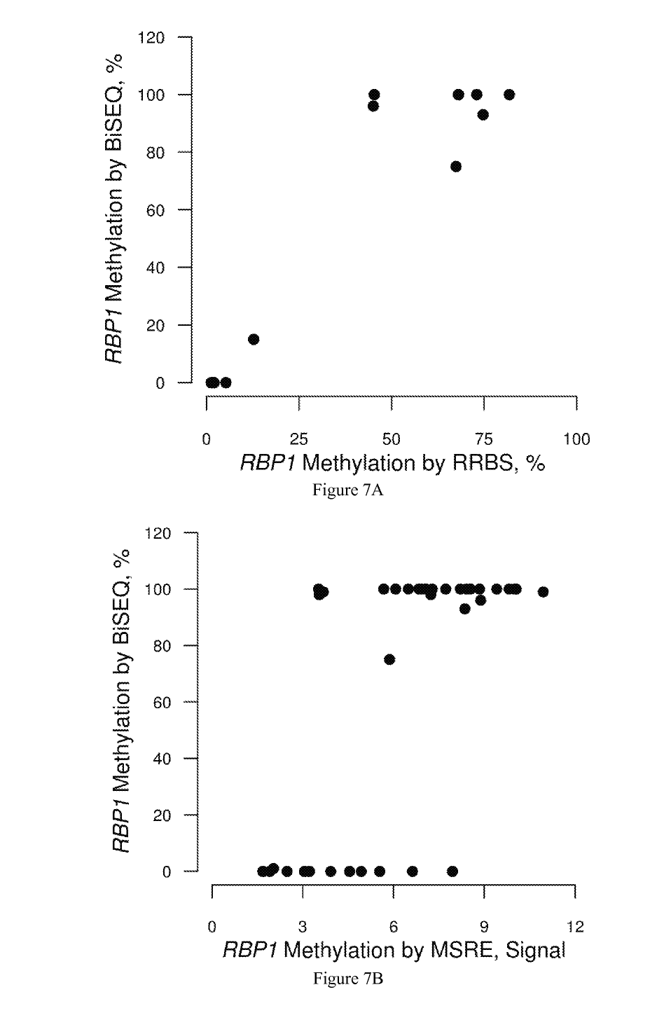

Given that RBP1 was found to be hypermethylated in the three datasets, correlated with decreased gene expression, and has been reported in the literature to be involved in the synthesis of the transcription regulator ATRA, RBP1 methylation was evaluated by gene-specific BiSEQ in the Total Cohort of 198 glioma patients with available frozen or paraffin-embedded tissues. All 198 patients in the Total Cohort had RBP1 methylation determined by BiSEQ and included the 41 patients in the initial methylation screen (10 by RRBS and 31 by MSRE). RBP1 was hypermethylated in 76 of 79 IDH1 mutant tumors, 3 of 3 IDH2 mutant tumors, and 0 of 116 WT tumors (Table 2). The RBP1 promoter was found by BiSEQ to be either essentially fully methylated or fully unmethylated across the 21 CpG sites evaluated (FIG. 6). Also, in samples with BiSEQ and RRBS/MSRE data, the results from the techniques were concordant, supporting the overall accuracy of the RRBS/MSRE dataset (FIGS. 7A-7B). These data show that both IDH1 and IDH2 mutations are highly correlated with RBP1 hypermethylation in the 198 gliomas analyzed (Spearman R²=0.94, 95% confidence interval=0.92 to 0.95, P<0.001).

### RBP1 Hypermethylation and CRBP1 Regulation

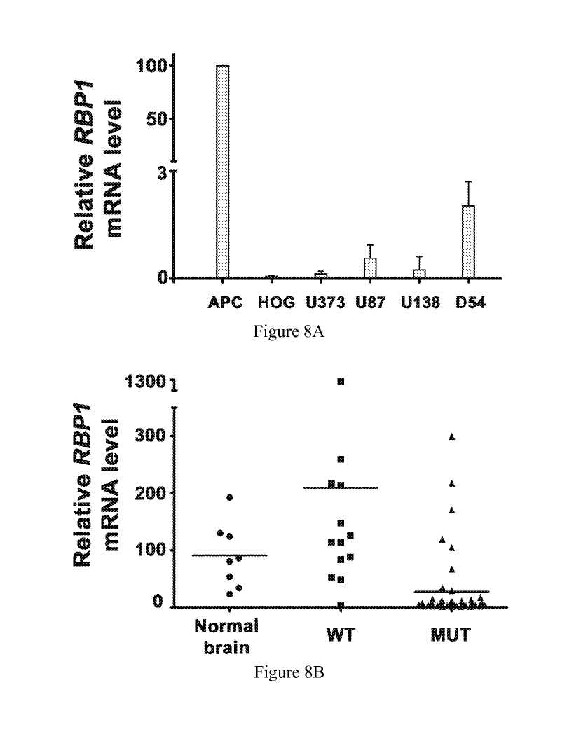

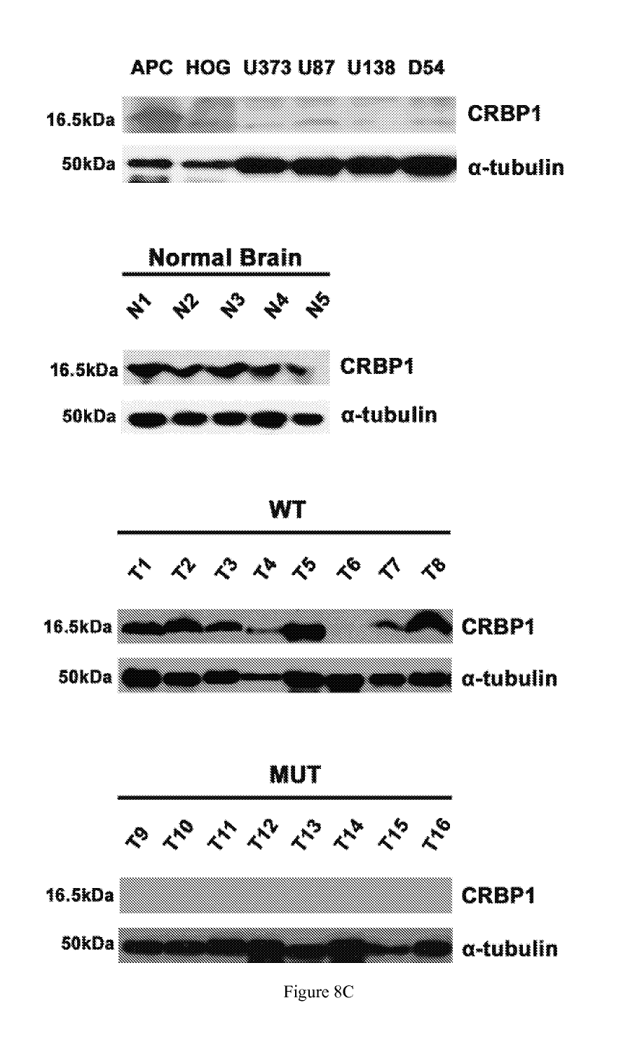

To assess if RBP1 hypermethylation is associated with decreased CRBP1 expression, mRNA and protein expression were measured in various cell lines and glioma tumor samples by quantitative RT-PCR and Western blot. Non-transformed APC cells were unmethylated for RBP1, and CRBP1 expression in APC cells was used as the reference. All of the tested glioma cells lines demonstrated RBP1 promoter methylation and decreased CRBP1 mRNA, despite being wild-type (FIG. 8A). This data appears consistent with the fact that the tested cell lines are immortalized cell lines. Specifically, experimental data (not shown) suggests that the RBP1 promoter in glioma derived cells become methylated after, i.e. as a result of, cell culturing regardless of IDH1 genotype (i.e. IDH1/IDH2 MUT or WT status). Also, quantitative RT-PCR and Western blot were performed on 12 WT, 43 IDH1 MUT, and 1 IDH2 MUT glioma samples using available frozen tissue. In all 56 samples, RBP1 was unmethylated in WT and methylated in IDH1/IDH2 MUT (data not shown). RBP1 mRNA levels from 7 normal brain samples were used as controls. FIG. 8B shows that IDH1/IDH2 MUT tumors (n=44) had statistically significantly decreased mRNA levels compared with WT tumors (n=12) (IDH1/IDH2 MUT tumors vs WT tumors: mean=17.93, SD=39.3 vs mean=208.9, SD=337.5, respectively; P<0.001). As shown in FIG. 8C, Western blot results were consistent with decreased CRBP1 protein expression in glioma cell lines. In particular, Western blots showed high CRBP1 protein expression in WT tumors (n=12), but essentially undetectable levels in MUT tumors (n=44) which was confirmed by densitometric analysis (relative CRBP1 in WT vs IDH1/IDH2 MUT tumors: mean=193.3, SD=224.2 vs mean=0.02, SD=0.15, respectively; P<0.001) (data not shown). This data demonstrates that in freshly derived tumor tissues, IDH1/IDH2 MUT is associated with RBP1 promoter methylation, decreased RBP1 mRNA levels, and decreased CRBP1 protein levels.

### Relationship Between Overall Survival and RBP1-Methylation Status

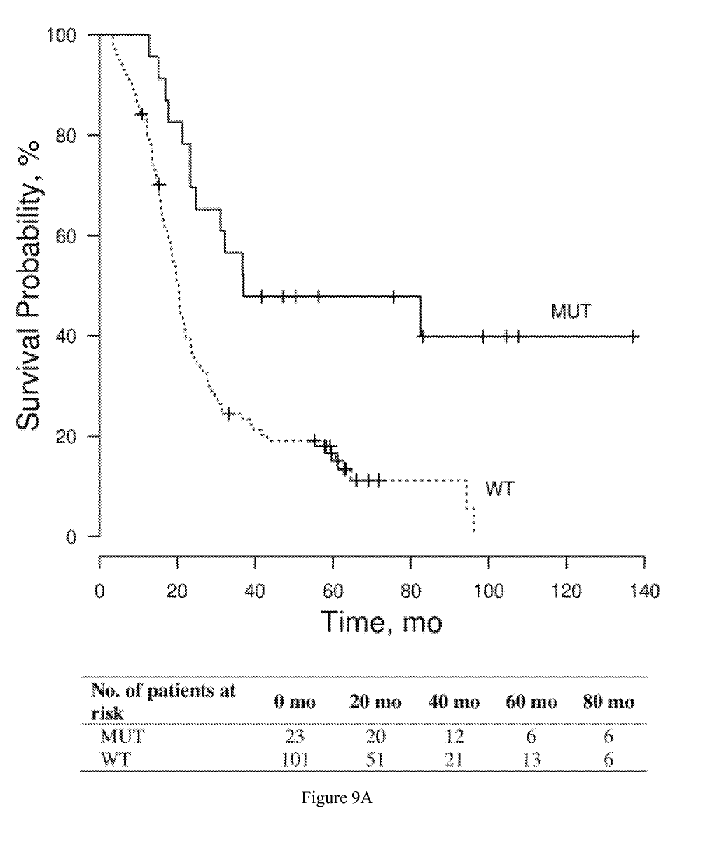

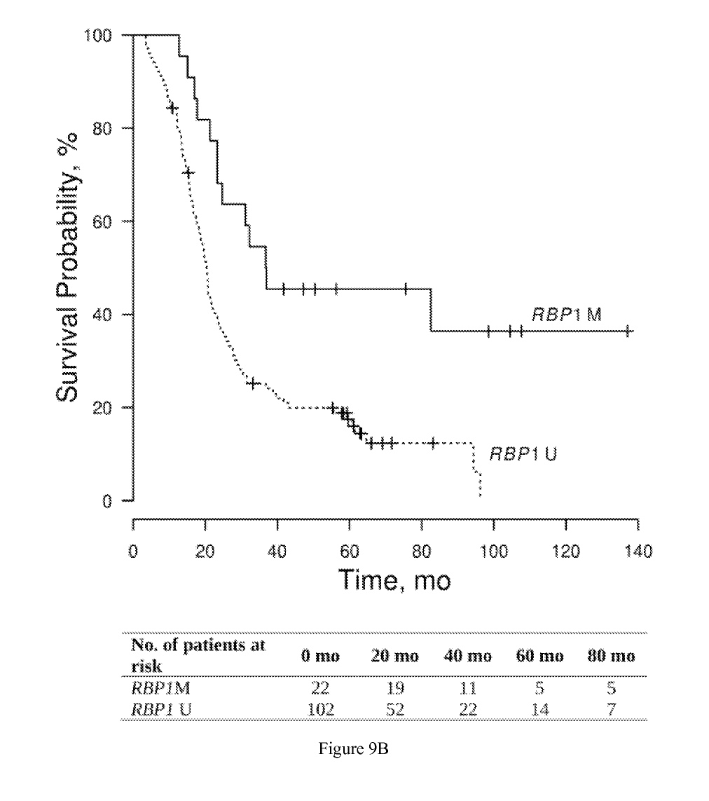

124 primary GBM patients were identified in the cohort who had treatment-naïve tumor samples and who had received standard chemo-radiation post-operatively (Stupp 2005). In the cohort, patients with WT GBM tumors (n=101) had a statistically significant decrease in median OS compared with patients with IDH1 or IDH2 MUT GBM tumors (n=23) (20.3 months vs 36.9 months, respectively; hazard ratio of death=2.91, 95% confidence interval=1.50 to 5.66, P=0.002) (FIG. 9A). As expected from the close association between RBP1 promoter methylation and IDH1 or IDH2 mutation, RBP1-unmethylated patients (n=102) had a statistically significantly decreased median OS of 20.3 months vs 36.8 months for RBP1-methylated patients (n=22; hazard ratio of death=2.48, 95% confidence interval=1.30 to 4.75, P=0.006) (FIG. 9B). As shown in Tables 5 and 6, multivariable analysis showed that either IDH1/IDH2 mutation or RBP1 methylation status is an independent predictor of OS when IDH1/IDH2 mutation or RBP1 methylation status was analyzed separately with the clinical variables age, sex, performance status, and extent of resection.

TABLE 5
Comparison of overall survival among the Total Cohort (n = 121) by
retinol binding protein-1 (RBP1) methylation status

| | No. of | | |
|---|---|---|---|
| Factor | patients | HR (95% CI) | P* |
| Age, y | 121 | 1.03 (1.01 to 1.04) | .006 |
| Sex | | | |
| Male | 70 | 1.06 (0.70 to 1.60) | .79 |
| Female | 54 | | |
| Karnofsky performance status | | | |
| ≦70 | 13 | 1.43 (0.75 to 2.70) | .28 |
| 80-100 | 111 | | |
| Resection | | | |
| Sub-total resection/biopsy | 70 | 1.57 (1.03 to 2.40) | .04 |
| Gross total resection | 57 | | |
| RBP1 methylation | | | |
| Methylated | 22 | 2.48 (1.30 to 4.75) | .006 |
| Unmethylated | 102 | | |

*Cox multivariable models were used to calculate the hazard ratios (HRs) and 95% confidence intervals (CIs). The Wald test was used to calculate two-sided P values.

TABLE 6
Comparison of overall survival among the Total Cohort (n = 121)
by isocitrate dehydrogenase (IDH1/IDH2) mutation status

| | No. of | | |
|---|---|---|---|
| Factor | patients | HR (95% CI) | P |
| Age, y | 121 | 1.02 (1.00 to 1.04) | .01 |
| Sex | | | |
| Male | 70 | 1.10 (0.73 to 1.67) | .65 |
| Female | 54 | | |
| Karnofsky performance status | | | |
| ≦70 | 13 | 1.42 (0.75 to 2.70) | .28 |
| 80-100 | 111 | | |
| Resection | | | |
| Sub-total resection/biopsy | 70 | 1.67 (1.09 to 2.57) | .02 |
| Gross total resection | 57 | | |
| IDH1/IDH2 | | | |
| WT | 101 | 2.91 (1.50 to 5.66) | .002 |
| MUT | 23 | | |

*Cox multivariable models were used to calculate the hazard ratios (HRs) and 95% confidence intervals (CIs). The Wald test was used to determine two-sided P values.
MUT = mutant;
WT = wild-type.

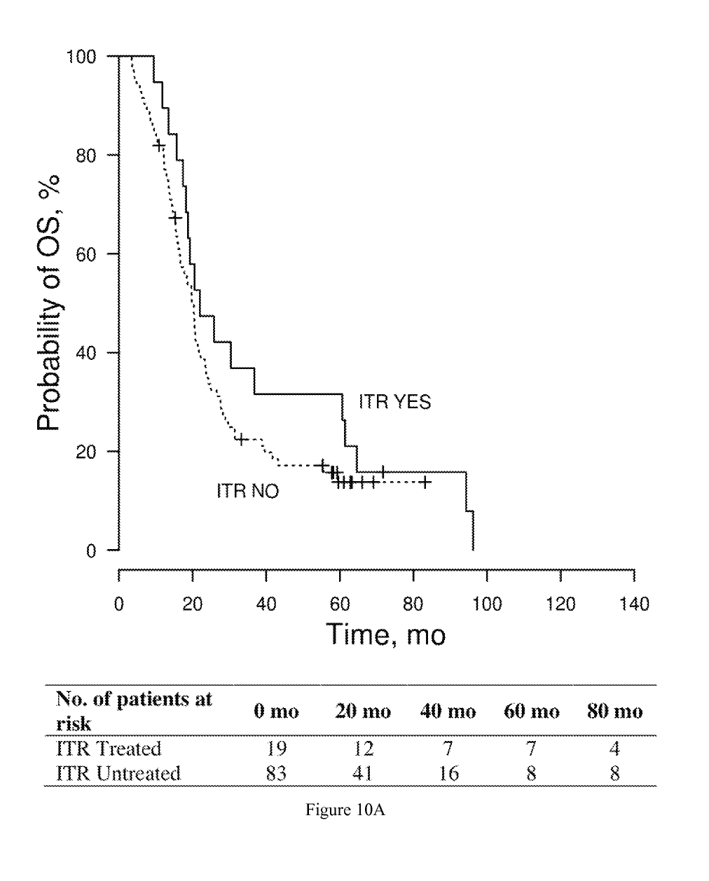

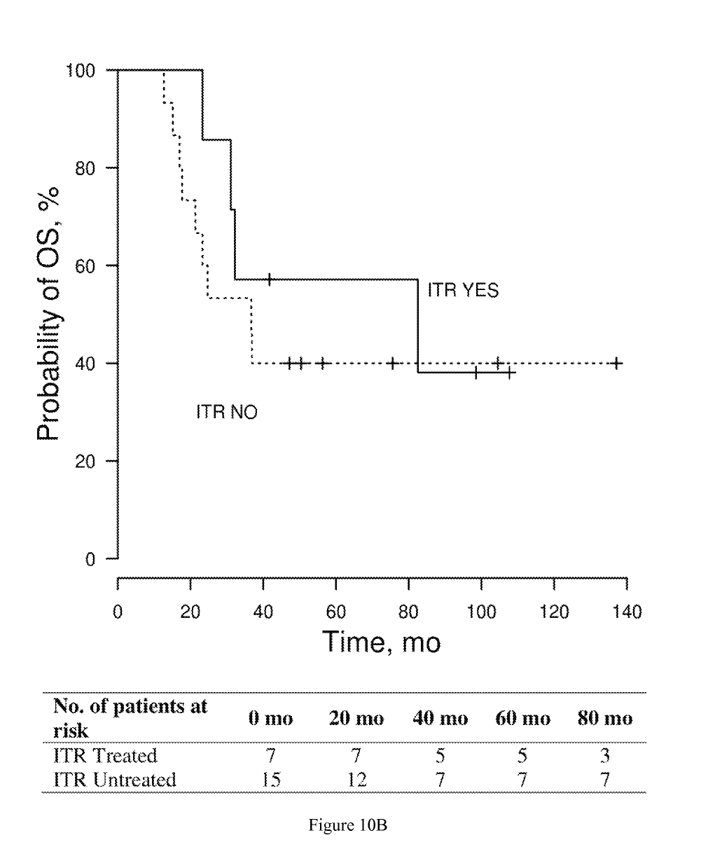

When patients were stratified by whether or not they received adjuvant isotretinoin with temozolomide, RBP1-unmethylated patients who were treated (n=19) and those who were not (n=83) had a similar median OS (22.0 months vs 19.8 months, respectively; P=0.25) (FIG. 10A). The median OS for RBP1-methylated patients who had received isotretinion (n=7) and untreated patients (n=15) was also similar (82.5 months vs 36.7 months, respectively; P=0.53) (FIG. 1 OB).

In summary, these experiments indicate that RBP1 promoter hypermethylation is found in nearly all IDH1/IDH2 MUT gliomas. Thus, RBP1 promoter hypermethylation can be used as a single diagnostic test to predict both IDH1 and IDH2 mutations at the same time instead of having to perform one assay for IDH1 and separate assay for IDH2. Therefore, the present invention provides a diagnostic test for IDH1/IDH2 mutations in a sample or subject which comprises detecting RBP1 promoter hypermethylation, wherein the presence of RBP1 promoter hypermethylation indicates an IDH1 and/or an IDH2 mutation.

As provided herein, RBP1 promoter hypermethylation is associated with decreased CRBP1 expression. In addition, glioma patients having RBP1 promoter hypermethylation were observed to have significantly higher survival rates than those not having RBP1 promoter hypermethylation. Thus, RBP1 promoter hypermethylation may be used as a biomarker for predicting the likely survival rate of a subject.

Hypermethylation of the RBP1 promoter can be tested by bisulfite sequencing, methylation specific PCR, or as a part of a methylation-sensitive microarray. RBP1 expression can be tested by RT-PCR, immunoblotting, or immunohistochemistry, and the like. Although the RBP1 promoter methylation status of a subject may be determined by any method know in the art, in some embodiments, the methods of the present invention are used.

### Retinoic Acid Treatment

A synthetic retinoic acid, isotretinoin (13-cis retinoic acid), has been used with little to only modest efficacy in unselected glioma patients (i.e. patients who have not been selected for any particular biomarker/mutation, including IDH1/IDH2 mutations) (Yung W K, et al. Clin Cancer Res 1996; 2(12):1931-5). It is noted that of all diffuse gliomas, roughly about 30% may be an IDH1/IDH2 MUT gliomas.

Despite the little to modest efficacy of retinoid therapy, retinoic acid supplementation may be particular effective in the treatment of IDH1/IDH2 MUT gliomas because RBP1 promoter hypermethylation is found in nearly all IDH1/IDH2 MUT gliomas and is associated with decreased CRBP1 expression and retinoic acid deficiency is hypothesized to contribute to the growth of IDH1 mutant tumors. In particular, it is hypothesized that because CRBP1 is involved in retinoic acid synthesis, dysregulation of retinoic acid metabolism may contribute to glioma formation and may play a role in the response to retinoid therapies.

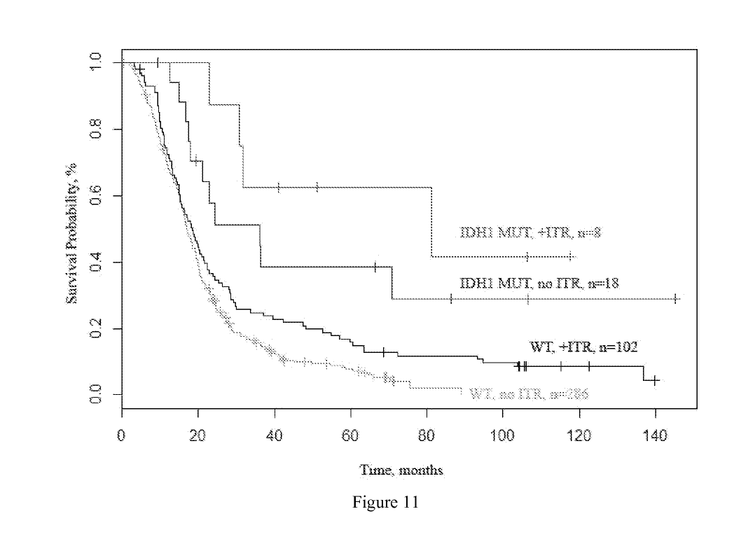

Thus, as set forth in FIG. 11, treatment with isotretinoin was found to significantly improve the overall survival of glioblastoma patients carrying the IDH1 mutation. Specifically, the median overall survival for IDH1 mutant glioblastoma patients treated with isotretinoin was 81 months as compared with 36 months for similar IDH1 mutant glioblastoma patients who were not treated with isotretinoin. Thus, treatment with one or more retinoids can significantly prolong the survival of patients with IDH1/IDH2 mutant gliomas. Therefore, in some embodiments, the present invention is directed to methods of treating IDH1/IDH2 mutant gliomas in subjects, which comprise administering to the subjects having been diagnosed with an IDH1 and/or an IDH2 mutation and/or RBP1 promoter hypermethylation one or more retinoids.

As used herein, a “retinoid” includes first generation retinoids such as retinoic acid, retinol, tretinoin, isotretinoin, and alitretinoin, second generation retinoids such as etretinate and acitretin, and third generation retinoids such as tazarotene, bexarotene, and Adapalene, and the like. Generally, first generation and third generation retinoids have a cyclic end group such as a 1,3,3-trimethylcyclohex-1-enyl group or a 1-methoxy-2,3,5-trimethylbenzenyl group, a polyene side chain and a polar end group. Thus, some retinoids according to the present invention include compounds having a 1,3,3-trimethylcyclohex-1-enyl group or a 1-methoxy-2,3,5-trimethylbenzenyl group, both of which may be substituted or unsubstituted, as the cyclic end group, a polyene side chain, and a polar end group.

In some embodiments of the present invention, RBP1 promoter hypermethylation in IDH1/IDH2 MUT gliomas may be used as a biomarker for determining whether a subject will likely respond to retinoid therapy, i.e. treatment with one or more retinoids. In addition, since IDH1 and/or IDH2 mutations are observed in other cancers, such as acute myelogenous leukemia (AML), RBP1 promoter hypermethylation may also be used as a biomarker for determining whether a subject suffers from a cancer associated with an IDH1 and/or an IDH2 mutation and/or whether the subject will likely respond to retinoid therapy. Thus, in some embodiments, the present invention is directed to methods of treating a cancer in a subject, which said cancer is associated with an IDH1 and/or an IDH2 mutation and/or RBP1 promoter hypermethylation, which comprise administering to the subject having been diagnosed with an IDH1 and/or an IDH2 mutation one or more retinoids. As used herein, a cancer “associated with an IDH1 and/or an IDH2 mutation” is one that coincides with an IDH1 and/or an IDH2 mutation in the subject. As used herein, a cancer “associated with RBP1 promoter hypermethylation” is one that coincides with RBP1 promoter hypermethylation. Cancers associated with an IDH1 and/or an IDH2 mutation and/or RBP1 promoter hypermethylation include IDH1/IDH2 MUT gliomas, acute myelogenous leukemia (AML), and the like.

In some embodiments, the treatment methods according to the present invention may further include radiation treatments and/or chemotherapy (e.g. treatment with temozolomide or the like) before, during, or after administration of the one or more retinoids. In some embodiments, a subject having a cancer, such as a glioma, is screened for having an IDH1 and/or IDH2 mutation and/or RBP1 promoter hypermethylation prior to treatment for the cancer. Then in the event that the subject has an IDH1 and/or IDH2 mutation and/or RBP1 promoter hypermethylation, the subject is subjected to treatment with one or more retinoids. The person performing the screening assay for the mutation and/or hypermethylated RBP1 promoter and/or the person who analyzes the results of the screening assay (e.g. laboratory technician) may be the same or different from the person administering the retinoid (e.g. doctor). In some embodiments, the subject to be treated is one who is at risk of having a cancer associated with an IDH1 mutation and/or an IDH2 mutation, e.g. an IDH1/IDH2 MUT glioma or AML. In some embodiments, the subject to be treated is one who has a genetic predisposition to cancers associated with IDH1 mutations and/or IDH2 mutations.

In some embodiments, the one or more retinoids are administered in a form of a pharmaceutical composition by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the cancer to be treated, and the chosen retinoid and formulation. In some embodiments, the retinoids are administered in a therapeutically effective amount. As used herein, a “therapeutically effective amount” is an amount which ameliorates the symptoms and/or pathology of the given cancer as compared to a control such as a placebo. A therapeutically effective amount may be readily determined by standard methods known in the art. The dosages to be administered can be determined by one of ordinary skill in the art depending on the clinical severity of the cancer, the age and weight of the subject, and other treatments the subject has or will be subjected to before, during, or after the treatment with one or more retinoids. It will be appreciated that the actual dosages will vary according to the particular retinoid or composition, the particular formulation, the mode of administration, and the particular site, host, and the cancer being treated. It will also be appreciated that the effective dosage used for treatment may increase or decrease over the course of a particular treatment. Optimal dosages for a given set of conditions may be ascertained by those skilled in the art using conventional dosage-determination tests in view of the experimental data for a given peptide or composition. Changes in dosage may result and become apparent by standard diagnostic assays known in the art. In some conditions chronic administration may be required.

As evidenced by the experiments above, the consequence of RBP1 promoter methylation is decreased function of the RBP1 gene, thus decreased RBP1 messenger RNA, decreased CRBP1 protein, and possibly decreased retinoic acid. Thus, in some embodiments, the present invention is directed to methods of detecting the presence of an isocitrate dehydrogenase mutation in a sample obtained from a subject which comprise measuring the amount of RBP1 messenger RNA and/or the amount of CRBP1 protein and/or the amount of retinoic acid in the sample. If the measured amount is less than that which is considered normal for WT samples, the presence of the mutation is indicated. If the mutation is indicated, the subject may be treated with one or more retinoids.

To the extent necessary to understand or complete the disclosure of the present invention, all publications, patents, and patent applications mentioned herein are expressly incorporated by reference therein to the same extent as though each were individually so incorporated. It should also be noted that the alternative language used herein is merely used to reduce the number of pages and claims. Thus, alternative language in the claims and description should be interpreted as if each alternative is individually and separately recited.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. An assay for detecting the presence of an isocitrate dehydrogenase mutation in a sample obtained from a subject which comprises
determining the methylation status of all or part of the retinol-binding protein 1 (RBP1) promoter in the sample, wherein the presence of the isocitrate dehydrogenase mutation is indicated where the methylation status is hypermethylated.

2. The assay according to claim 1, which comprises characterizing the methylation status to be hypermethylated where the all or part of the RBP1 promoter is significantly more methylated than that of the corresponding part of a standard which is a methylation profile of a wild type RBP1 promoter or a consensus methylation profile of the RBP1 promoters obtained from a plurality of normal subjects.

3. The method according to claim 2, wherein the methylation status is characterized as being hypermethylated where the overall methylation of the all or part of the RBP1 promoter is methylated by 50% or more than that of the corresponding part of the standard.

4. The assay according to claim 1, wherein the RBP1 promoter is located on chromosome 3 at 140740839-140741418.

5. The assay according to claim 1, wherein the part of the RBP1 promoter comprises, consists essentially of, or consists of one or more of the CpG sites of the RBP1 promoter.

6. The assay according to claim 1, wherein the part of the RBP1 promoter comprises, consists essentially of, or consists of CpG sites 5-25 of the CpG island located on chromosome 3 at 140740839-140741418.

7. The assay according to claim 1, wherein the part of the RBP1 promoter comprises, consists essentially of, or consists of at least 3, preferably at least 5, more preferably at least 10 CpG sites selected from the group consisting of CpG sites 14-24, 26-31, 39-45 and 59 of the CpG island located on chromosome 3 at 140740839-140741418.

8. The assay according to claim 1, wherein the part of the RBP1 promoter comprises, consists essentially of or consists of at least 6, preferably at least 10, more preferably at least 15 CpG sites selected from the group consisting of CpG sites 1-5, 14-24, 26-31, 39-45 and 59 of the CpG island located on chromosome 3 at 140740839-140741418.

9. The assay according to claim 1, wherein the part of the RBP1 promoter comprises, consists essentially of, or consists of at least 10, preferably at least 15, more preferably at least 20 CpG sites selected from the group consisting of CpG sites 1-5, 14-24, 26-31, 39-45 and 59-62 of the CpG island located on chromosome 3 at 140740839-140741418.

10. The assay according to claim 4, which comprises characterizing the methylation status as being hypermethylated where the overall methylation of the all or part of the RBP1 promoter is methylated by 50% or more than that of the corresponding part of a standard which is a methylation profile of a wild type RBP1 promoter or a consensus methylation profile of the RBP1 promoters obtained from a plurality of normal subjects.

11. The assay according to claim 1, wherein when one or more of the CpG sites 15, 21, 24, 29, 30, 40, 44 or 45 of the CpG island located on chromosome 3 at 140740839-140741418 are methylated, the methylation status is designated as hypermethylated.

12. The assay according to claim 1, which further comprises designating the presence of the isocitrate dehydrogenase mutation where the methylation status is hypermethylated.

13. The assay according to claim 1, wherein the isocitrate dehydrogenase mutation is an isocitrate dehydrogenase 1 (IDH1) mutation or an isocitrate dehydrogenase 2 (IDH2) mutation.

14. The assay according to claim 1, which comprises modifying all or part of the retinol-binding protein 1 (RBP1) promoter in the sample to make the methylated cytosines of CpG dinucleotides distinguishable from the unmethylated cytosines of CpG dinucleotides.

15. The assay according to claim 14, wherein the modification comprises subjecting all or part of the RBP 1 promoter in the sample to (a) a bisulfite that converts the unmethylated cytosines to uracils, (b) a restriction enzyme that selectively cleaves the unmethylated cytosines, (c) a label specific for either the unmethylated cytosines or the methylated cytosines, or (d) a combination thereof.

16. The assay according to claim 1, wherein the methylation status is determined using reduced representation bisulfite sequencing (RRBS).

17. A method of providing a diagnosis and/or prognosis to a subject having a cancer, which comprises
giving the diagnosis and/or prognosis to the subject based on the presence or absence of an isocitrate dehydrogenase mutation and/or the methylation status of all or part of the retinol-binding protein 1 (RBP1) promoter in a sample obtained from the subject,
wherein the diagnosis is that the cancer may likely be treated with one or more retinoids where the isocitrate dehydrogenase mutation is present and/or the all or part of the RBP1 promoter is hypermethylated, and
wherein the prognosis is that the subject will have an estimated time of survival that will likely be increased with treatment with one or more retinoids where the isocitrate dehydrogenase mutation is present and/or the all or part of the RBP1 promoter is hypermethylated.

18. A method of treating a subject having a cancer having been determined to be associated with an isocitrate dehydrogenase mutation and/or associated with hypermethylation of all or part of the retinol-binding protein 1 (RBP1) promoter, which comprises
administering to the subject one or more retinoids.

19. The method according to claim 17, wherein the presence or association with the isocitrate dehydrogenase mutation and/or the methylation status of the all or part of the RBP1 promoter is or was determined using an assay.

20-23. (canceled)
